(19) United States
(12) Patent Application Publication
(10) Pub. No.: US 2013/0280220 A1
(43) Pub. Date: Oct. 24, 2013

(54) CHIMERIC ANTIGEN RECEPTOR FOR BISPECIFIC ACTIVATION AND TARGETING OF T LYMPHOCYTES

(71) Applicants: Nabil Ahmed, Houston, TX (US); Zakaria Grada, Houston, TX (US); Meenakshi Hedge, Houston, TX (US); Matthew Baker, Bellaire, TX (US); Donald R. Shaffer, Houston, TX (US)

(72) Inventors: Nabil Ahmed, Houston, TX (US); Zakaria Grada, Houston, TX (US); Meenakshi Hedge, Houston, TX (US); Matthew Baker, Bellaire, TX (US); Donald R. Shaffer, Houston, TX (US)

(21) Appl. No.: 13/788,267

(22) Filed: Mar. 7, 2013

Related U.S. Application Data
(60) Provisional application No. 61/635,983, filed on Apr. 20, 2012.

(51) Int. Cl. C12N 15/85; A61K 45/06; A61K 35/14

(52) U.S. Cl. CPC C12N 15/85; A61K 35/17; A61K 45/06
USPC 424/93.21; 435/325; 435/320.1; 435/455

(57) ABSTRACT

Embodiments of the invention include methods and compositions related to improved cells encoding a chimeric antigen receptor that is specific for two or more antigens. In certain aspects the receptor encompasses two or more non-identical antigen recognition domains. The antigens are tumor antigens, in particular embodiments.

### CROSS-REFERENCE TO RELATED APPLICATIONS

This application claims priority to U.S. Provisional Application 61/635,983 filed on Apr. 20, 2012, which is incorporated by reference herein in its entirety.

### TECHNICAL FIELD

Embodiments of the invention include at least the fields of immunology, cell biology, molecular biology, and medicine, including cancer medicine.

### BACKGROUND OF THE INVENTION

Chimeric antigen receptors (CARs) are artificial molecules that redirect the specificity of T cells to predetermined antigens (Pule et al., 2003). Prototypic single chain CARs were first described in a study by Eshhar and colleagues in 1993, in which specific activation and targeting of T cells was mediated through molecules consisting of a target-antigen-specific antibody domain and the γ- or ζ-signaling subunits of the Fc epsilon receptor or T-cell receptor CD3 complex, respectively (Pule et al., 2003; Eshhar et al., 1993). Since then, many groups have devised CAR molecules with single tumor-directed specificities and enhanced signaling endodomains (Ahmed et al., 2007; Brentjens et al., 2003; Pule et al., 2004; Savoldo et al., 2007); CAR T cell-based clinical trials are currently underway, with early results being highly promising (Dotti et al., 2009; Pule et al., 2008; Kalos et al., 2011; Porter et al., 2011; Study of Administration of CMV-specific Cytotoxic T Lymphocytes Expressing CAR Targeting HER2 I Patients with GBM, 2011; Her2 and TGFBeta in Treatment of Her2 Positive Lung Malignancy, 2011).

Rendering an individual T cell bispecific could have substantial functional implications that would likely translate into major therapeutic benefits. Down-regulation or mutation of target antigens is commonly observed in cancer cells, creating antigen loss escape variants; a bispecific T cell could thus offset tumor escape (Dunn et al., 2004). Furthermore, this bi-specificity could enable simultaneous targeting of tumor cells and elements in the tumor microenvironment thereby augmenting T-cell activation and function by increasing avidity and by broadening their therapeutic reach (Weijtens et al., 2000). To accomplish such bispecificity, the inventors constructed a CAR in which two distinct antigen recognition domains are present in tandem on a single transgenic receptor.

The folding of an amino acid chain into highly organized, biologically functional three-dimensional protein structures, such as a CAR, continues to be a challenge in the design of novel protein molecules (Buchner et al., 2011). In particular, protein misfolding, mispairing and malfunction/dysfunction, have traditionally impeded attempts at production of molecules with multiple specificities (Kuhlman and Baker, 2004). Advances in computational modeling methods, through characterization of the underlying energy landscapes as well as the dynamics of the polypeptide chains, have made structure prediction, analysis and design of a novel protein molecule, such as a tandem CAR, more feasible (Park et al., 2004; Perez-Aguilar and Saven, 2012; Samish et al., 2011). Furthermore, docking routines have recently made it possible to predict, with high accuracy, the interface between two candidate molecules in a manner that could help to elucidate their functionality (Wodak, 2007; Kiel et al., 2008).

The inventors used computational modeling tools to guide the design and construction of a novel single CAR molecule that can mediate bispecific activation and targeting of T cells. This tandem CAR (TanCAR), recognizes each target molecule individually, and facilitates synergistic activation and functionality when both are encountered simultaneously. Thus, the present invention provides important therapeutic advances in the art.

### BRIEF SUMMARY OF THE INVENTION

The present invention is directed to methods and compositions related to improved immunogenic therapeutic compositions that comprise chimeric antigen receptors (CARs). The present invention is directed to methods and compositions related to cell therapy. In particular embodiments, the cell therapy is for an individual in need of cell therapy, such as a mammal, including a human. The cell therapy may be suitable for any medical condition, although in specific embodiments the cell therapy is for cancer, including cancer having solid tumors.

In certain embodiments, the cancer may be of any kind and of any stage. The individual may be of any age or either gender. In specific embodiments, the individual is known to have cancer, is at risk for having cancer, or is suspected of having cancer. The cancer may be a primary or metastatic cancer, and the cancer may be refractory to treatment. In specific embodiments, the cancer is leukemia, lymphoma, myeloma, breast, lung, brain, colon, kidney, prostate, pancreatic, thyroid, bone, cervical, spleen, anal, esophageal, head and neck, stomach, gall bladder, melanoma, non-small cell lung cancer, and so forth, for example, such as various types of primary and secondary brain and liver cancers. In particular aspects, the cancer expresses one or more tumor antigens, although upon identification of a type of cancer in an individual, the presence of the particular tumor antigen(s) may or may not be verified.

In certain embodiments of the invention, the invention concerns methods and compositions related to therapeutic cells, including therapeutic immune system cells such as tumor-specific cytotoxic T lymphocytes. The cells may be NK cells or NKT cells, in some cases, however, other cellular elements with the capability of inducing an effector immune response are encompassed in the invention. The cells express at least one non-endogenous receptor that targets two or more particular tumor antigens, and in at least some cases, the receptor comprises a scFv.

Embodiments of the invention include a tandem chimeric antigen receptor that mediates bispecific activation and targeting of T cells. Although the present disclosure refers to bispecificity for the CAR, in some cases the CARs are able to target three, four, or more tumor antigens. Given that single agents in cancer therapy fail to cure tumors while multiple agents achieve substantial responses (or cure), targeting multiple antigens using CAR T cells of the present invention results in (1) enhanced T cell activation, (2) effectively offsetting tumor escape by antigen loss, and (3) enhancing tumor control by capturing more tumor bulk and a collective action of the above former two effects.

In certain aspects to the invention, there are bispecific tandem chimeric antigen receptor (TanCAR) that includes two targeting domains. In certain aspects to the invention, there is multispecific tandem chimeric antigen receptor (TanCAR) that includes three or more targeting domains. TanCARs of the invention augment T-cell activation and function by increasing avidity and by broadening their therapeutic reach. This allows for (1) targeting multiple modestly expressed antigens, (2) targeting various tumors using the same cellular product that has a broad specificity and allows for (3) better toxicity profile because a less intensely signaling CAR could achieve the same results by virtue of multiple specificity.

The TanCAR of the present invention may target two or more tumor antigens of any kind. Exemplary tumor antigens include one or more of CD19, CD20, CD22, k light chain, CD30, CD33, CD123, CD38, ROR1, ErbB2, ErbB3/4, EGFr vIII, carcinoembryonic antigen, EGP2, EGP40, mesothelin, TAG72, PSMA, NKG2D ligands, B7-H6, IL-13 receptor α 2, MUC1, MUC16, CA9, GD2, GD3, HMW-MAA, CD171, Lewis Y, G250/CALX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, and/or TEM8.

In some embodiments of the invention, there is a bispecific TanCAR that targets HER2 and another tumor antigen. In some cases there is a bispecific TanCAR that targets IL13R-alpha2 and another tumor antigen. In some cases there is a bispecific TanCAR that targets VEGF-A and another tumor antigen. In some cases there is a bispecific TanCAR that targets Tem8 and another tumor antigen. In some cases there is a bispecific TanCAR that targets FAP and another tumor antigen. In some cases there is a bispecific TanCAR that targets EphA2 and another tumor antigen. In some cases there is a bispecific TanCAR that targets CD19 and another tumor antigen. In certain embodiments of the invention, there is a bispecific TanCAR that targets one or more, two or more, three or more, or four or more of the following tumor antigens: HER2, IL13R-alpha2, VEGF-A, Tem8, FAP, EphA2, or CD19.

In particular embodiments, there is a bispecific TanCAR that targets HER2 and IL13R-alpha2 (HER2—IL13Ra2 TanCAR) for the treatment of glioblastoma, for example, although other cancers may also be targeted.

In certain embodiments, there is targeting of the tumor complex, wherein multi-specificity enables simultaneous targeting of tumor cells and elements in the tumor microenvironment. In specific aspects such a composition includes a HER2 and VEGF-A specific TanCAR, for example. In some embodiments, Tem8 and/or FAP are targeted in the invention and, therefore have TanCARs with one or both of them.

In embodiments of the invention, there is a T lymphocyte, or pluralities thereof, comprising a bi-specific or multi-specific chimeric antigen receptor, said receptor comprising two or more non-identical antigen recognition domains. In specific embodiments, the antigen recognition domains are further defined as an exodomain comprising a single chain variable fragment specific for a first antigen and a single chain variable fragment specific for a second antigen. In some embodiments, the antigens to which the chimeric antigen receptor is bi-specific or multi-specific are not present on the same endogenous cells. In some embodiments, the antigens to which the chimeric antigen receptor is bi-specific or multi-specific are present on the same endogenous cells. In specific cases, at least one of the antigens that the chimeric antigen receptor recognizes is present on the surface of a cancer cell. In some embodiments, at least one of the antigens that the chimeric antigen receptor recognizes is present in a tumor microenvironment. In some aspects of the invention, at least one of the antigens to which the chimeric antigen receptor recognizes is a growth factor. The antigen may be VEGF-A, Tem8 or FAP, in at least some cases.

In certain aspects for the cells of the invention, the length between the antigen recognition domains on the receptor is between about 5 and about 30 amino acids. At least one of the antigens recognized by the antigen recognition domains may be selected from the group consisting of HER2, CD19, IL13R-alpha2, Tem8, FAP, EphA2 and VEGF-A. In some cases, the receptor further comprises a signaling endodomain of a costimulatory molecule selected from the group consisting of CD 28, 41BB, OX40 and zeta chain of the T cell receptor.

In some embodiments of the invention, there is a substrate comprising a plurality of cells, including T cells, NK cells, and NKT cells. T lymphocytes may be employed the invention. In certain embodiments, a plurality comprises T lymphocytes that recognize different groups of antigens.

In some embodiments of the invention, there is an expression vector encoding a bi-specific or multi-specific chimeric antigen receptor, said receptor comprising two or more non-identical antigen recognition domains. In some cases, the vector is an integrating vector or not an integrating vector. The vector may be a lentiviral vector, a retroviral vector, an adenoviral vector, an adeno-associated viral vector, a plasmid, or RNA.

In some embodiments, there is a method of producing a T lymphocyte comprising a bi-specific or multi-specific chimeric antigen receptor, said receptor comprising two or more non-identical antigen recognition domains, comprising the step of transducing a T lymphocyte with a vector as described herein.

In some embodiments, there is a method of killing a cancer cell in an individual, comprising the step of providing to the individual a therapeutically effective amount of a therapeutic cell of the invention, including an effector cell, such as a T cell, NK cell, NKT cell, or T lymphocyte of the invention, for example. The individual may have breast cancer, lung cancer, brain cancer, prostate cancer, pancreatic cancer, ovarian cancer, colon cancer, liver cancer, thyroid cancer, skin cancer, testicular cancer, gall bladder cancer, esophageal cancer, spleen cancer, or cervical cancer, for example. In specific cases, the cancer cell expresses at least one of the antigens. In certain aspects of the invention, the cancer cell is carcinoma or sarcoma. Any method of the invention may further comprise the step of delivering to the individual an additional cancer therapy, such as surgery, radiation, hormone therapy, chemotherapy, immunotherapy, or a combination thereof, for example.

In embodiments of the invention there is a cell comprising a chimeric antigen receptor (CAR) comprising two or more non-identical antigen recognition domains. The CAR may be further defined as comprising an exodomain comprising an antigen recognition domain specific for a first tumor antigen and an antigen recognition domain specific for a second tumor antigen. In particular embodiments, the two or more antigens are configured in the CAR in a tandem arrangement. In specific embodiments, at least one of the first and second tumor antigens is specific for an antigen present on a cancer cell surface, such as HER2, CD19, IL13R-alpha2, Tem8, MUC1, PSMA or EphA2, for example. In specific cases, at least one of the first and second tumor antigens is specific for an antigen present in a tumor microenvironment. The first or second tumor antigen may be specific for VEGF-A, Tem8 or FAP.

In some embodiments, the first tumor antigen is specific for an antigen present on a cancer cell surface and the second tumor antigen is present in a tumor microenvironment. The first tumor antigen, second tumor antigen, or both may be specific for a growth factor. In some cases, there is a linker region between the two non-identical antigen recognition domains, such as the linker region being between 5 and 30 amino acids. The linker region may be comprised of glycine, serine, or both.

In specific embodiments, the CAR further comprises a signaling endodomain of a costimulatory molecule selected from the group consisting of CD 28, 41BB, OX40 and zeta chain of the T cell receptor. In some cases, the two non-identical antigen recognition domains are HER2 and VEGF-A or HER2 and CD19. In specific aspects, the two non-identical antigen recognition domains are selected from the group consisting of HER2, CD19, IL13R-alpha2, Tem8, FAP, EphA2 and VEGF-A. In certain embodiments, the cell is a T cell, a NK cell, or a NKT cell.

In embodiments of the invention, there is an expression vector encoding a CAR comprising two or more non-identical antigen recognition domains. In some embodiments, the vector is further defined as a lentiviral vector, a retroviral vector, an adenoviral vector, an adeno-associated viral vector, a plasmid, or RNA.

In embodiments of the invention, there is a method of producing a cell of the invention, comprising the step of transducing a T lymphocyte (or T cell or NK cell or NKT cell) with an expression vector that encodes a CAR comprising two or more non-identical antigen recognition domains.

In embodiments of the invention, there is a method of killing a cancer cell in an individual, comprising the step of providing to the individual a therapeutically effective amount of cells of the invention.

In particular embodiments of any method of the invention, the individual has breast cancer, lung cancer, brain cancer, prostate cancer, pancreatic cancer, ovarian cancer, colon cancer, liver cancer, thyroid cancer, skin cancer, testicular cancer, gall bladder cancer, esophageal cancer, spleen cancer, cervical cancer, or primary or secondary malignancies of the nervous system. Methods of the invention may further comprise the step of delivering to the individual an additional cancer therapy, such as surgery, radiation, hormone therapy, chemotherapy, immunotherapy, or a combination thereof. In specific embodiments, when the CAR is specific at least for HER2, the individual is provided an additional HER2 therapy, and in some cases, when the CAR is specific at least for VEGF-A, the individual is provided an additional VEGF-A therapy.

In embodiments of the invention, there is a kit comprising cells comprising a chimeric antigen receptor (CAR) comprising two or more non-identical antigen recognition domains and/or expression vector encoding a CAR comprising two or more non-identical antigen recognition domains.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows designing of a bi-specific tandem chimeric antigen receptor (TanCAR) molecule. A cartoon of the proposed chimeric antigen receptor molecule engaging the two exemplary targets; HER2 and CD 19, is provided.

FIG. 2 demonstrates docking platforms predict favorable binding potential of TanCAR to target molecules. Compilation of structure and docking data of A) hypothetical structure of both FRP5-derived scFv and the CD19-specific scFv joined with a 20 amino acid Gly-Ser linker, B) most favorable docking models of FRP5-derived scFv and the distal 200 amino acid residues of the extracellular domain of HER2; C) most favorable docking models of CD19 scFv and the extracellular domain of CD19; combined docking of D) HER2 and E) CD19 and the TanCAR and F) collective favorable modeling of the simultaneous docking of the TanCAR to both HER2 and CD19.

FIG. 3 shows construction and surface expression of the TanCAR molecule: A) pSFG vector construct encoding the TanCAR; B) detection of the surface expression of the TanCAR using a Fab-specific antibody and FRP5-specific HER2-Fc protein on 293T cells; and C) on T cells. See FIG. 7 for description of the labeling strategy.

FIG. 4 provides that the TanCAR T cells distinctly recognized individual target molecules. A) Flow cytometry of the surface expression of the target antigens, HER2 and CD19, on a panel of human cancer cell lines used for functional testing; B) cytotoxicity assay showing recognition and killing of HER2 positive Daoy cells and efficient blocking of this lysis using a soluble HER2 fragment; C) similarly TanCAR T cells recognized CD19 positive Raji cells and this lysis was blocked using the CD19 Ab 4G7; D) in cocultures, TanCAR T cells secreted IFN-γ as well as IL-2 upon encounter of HER2— and CD19-positive target cells above the non-transduced T cell control (NT). No cytokines were secreted in coculture with the HER2 CD19 null target cell MDA-MB-468.

FIG. 5 demonstrates preserved TanCAR T cell-induced cytolysis in an exemplary model of antigen loss and enhanced cytolytic function upon simultaneous recognition of two antigens. A) The inventors modeled the scenario in which tumor cells down regulate the target antigen, by blocking HER2 in CD19-induced (D+) and CD19 null (D-) Daoy.TET.CD19 cells using a soluble HER2 fragment. While soluble HER2 successfully induced substantial blocking of HER2 mediated killing in D-cells at various tumor to T cell ratios, it could only induce partial decrease in the cytolytic effect of TanCAR T cells in D+. p-values were significant at all tumor to T cell ratios. B) In cytotoxicity assays, we saw consistently higher killing after the induction of CD19 at various tumor to T cell ratios. This was synergistic; with an exponential trend following a higher order equation; and was more prominent in higher tumor to T cell ratios (right panel). C) Similarly, induction of CD19 (D+) in Daoy.TET.CD19 and T cell cocultures resulted in more than four-fold increase in IFNγ release as detected by ELISA (p<0.01).

FIG. 6 shows simultaneous targeting of two antigens enhances the in vivo antitumor activity of adoptively transferred TanCAR T cells. A) Daoy.TET.CD19 xenografts were established for 3 weeks in the flanks of SCID mice, then animals were randomized into four groups. Administration of PBS into the tumor and/or systemic doxycycline induced minimal or no alteration of the tumor growth pattern. By contrast, treatment with TanCAR T cells resulted in a significant delay in tumor progression that was further enhanced by induction of CD19 expression in the D+group. B) Kaplan-Meier survival curve: Survival analysis performed 60 days after 60 days after the PBS or T cells injection. Mice treated with TanCAR T cells had a significantly longer survival probability in comparison to control mice. Furthermore, induction of CD19 by the administration of doxycycline resulted in enhanced antitumor activity of adoptively transferred TanCAR T cells.

FIG. 7 demonstrates binding of anti-HER2 antibody FRP5 to peptide arrays. Residues 1 to 300 of human HER2 precursor protein (uniprot accession number P04626) were synthesized as 283 18mer peptides with 17 residues overlap (peptides B-1 to I-31) on a cellulose membrane by automated parallel peptide synthesis. The membrane was incubated with FRP5 antibody, and binding was analyzed with HRP-coupled secondary antibody and chemi-luminescent detection. Major interactions were found with overlapping peptides B-32 to B-35 encompassing HER2 residues 32 to 52, D-8 to D-28 encompassing HER2 residues 80 to 117, E-18 to E-21 encompassing HER2 residues 126 to 146, E-32 to E-36 encompassing HER2 residues 140 to 161, F-19 to F-21 encompassing HER2 residues 163 to 182, and F-29 encompassing HER2 residues 173 to 190. Specificity of binding was confirmed by reprobing the membrane with secondary antibody alone or HER2-specific antibody trastuzumab that binds to a juxtamembrane HER2 epitope outside of the 300 residues synthesized.

FIG. 8 shows surface expression of the TanCAR was tested using a HER2 scFv (FRP5)-specific method by incubation with a soluble HER2.Fc fragment followed by a human Fc specific FITC-labeled antibody. Alternatively, APC-conjugated Fab-specific antibody was used to detect either HER2 scFv (FRP5) or CD19 scFv.

FIG. 9 demonstrates a tetracycline inducible system to conditionally express a truncated non-signaling CD19 molecule on Daoy cells (Daoy.TET.CD19).⁴³ In the presence of Doxycyline, 60-85% of the endogenous HER2 positive Daoy.TET.CD19 cells expressed CD 19 and the exemplary reporter gene mCherry.

FIG. 10 illustrates that CARs are synthetic molecules that consist of an extracellular receptor ectodomain that contains the heavy and light chain variable regions of a monoclonal antibody joined to a transmembrane and a cytoplasmic signaling endodomain derived from the CD3-ζ chain and optionally costimulatory molecules such as CD28, OX40, or 4-1BB (Pule et al., 2003; Gross et al., 1989).

FIG. 11 demonstrates that guided by data from computational platforms of protein structure and docking, the inventors constructed a novel proof-of-concept CAR molecule by joining by a linker, in tandem, two single chain variable fragments (scFv) molecules specific for CD19 and HER2. This exodomain was tethered to a hinge and transmembrane and signaling domain.

FIG. 12 provides that HER2/VEGF-A TanCAR T cells could engage both antigen molecules simultaneously. This not only targets the cancer cell (HER2) but the supporting cellular elements of the tumor microenvironment which secrete VEGF-A and to which this vascular mitogen is tethered.

FIG. 13 shows that HER2/VEGF-A TanCAR could engage both HER2 (constitutively expressed) and VEGF-A (conditional) on hypoxic tumor cells. This results in improved T cell activation in hypoxic areas-where T cell function is usually compromised. Moreover, it is particularly advantageous in the context of low target expression of tumor antigens.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein embodiments which are disclosed and still obtain a like or similar result without departing from the spirit and scope of the invention.

As used herein, the term “tumor microenvironment” refers to any and all elements of the tumor milieu that creates a structural and or functional environment for the malignant process to survive and/or expand and/or spread.

Highly selective targeted T cell therapies are emerging as effective non-toxic modalities for the treatment of cancer. Malignancies are complex diseases where multiple elements contribute to the overall pathogenesis through both distinct and redundant mechanisms. Hence, targeting different cancer-specific markers simultaneously could result in better therapeutic efficacy. However, developing two separate cellular products for clinical use as combination therapy is impractical, owing to regulatory hurdles and cost.

In contrast, rendering an individual T cell bispecific could increase target cell selectivity, improve T-cell activation and offset tumor escape because of antigen loss. Here, the present invention provides a generally applicable approach in which T cells have been modified to express a novel tandem chimeric antigen receptor (TanCAR) that can distinctly recognize two tumor antigens simultaneously.

In specific embodiments, the design of the TanCAR was guided by systematic computational modeling and incorporates, in tandem, two single chain antibody variable fragments (scFv) tethered to a hinge, and transmembrane and signaling domains. Engagement of TanCAR by cognate ligands induced activation of T cells, which had effector activity against individual target antigens, and synergistic enhancement of functionality upon simultaneous ligation of both components. Antitumoral activity of TanCAR T cells was observed in an animal tumor model, demonstrating their utility for therapeutic application in human disease.

Thus, embodiments of the invention utilize a TanCAR as an artificial molecule that enables immune cells (T cells) to specifically and distinctly recognize and attack two cancer target molecules simultaneously. The CAR, is an artificial molecule that can be grafted onto T cells using genetic engineering technology to render them specific to a target of interest. The prototype such molecule consisted of two parts: one that projects outside the T cell to engage its target and the other extends inside it and is responsible for activation of the T cell killing machinery upon target engagement. The TanCAR, or Tandem Chimeric Antigen Receptor, has not one but two recognition domains, in tandem, projecting outside the T cells enabling a single T cell to recognize two molecules and attack them simultaneously.

TanCAR-grafted T cells by virtue of their duality are able to identify a) multiple cells expressing these target molecules and/or b) multiple target molecules on the same cell. This ability has substantial therapeutic implications. Growing evidence indicates that cancer cells can only live and grow if they succeed in creating congenial soil referred to as the tumor microenvironment. Cellular elements of the tumor microenvironment secrete tumor promoting factors that maintain such soil. Embodiments of the invention incude a TanCAR molecule to simultaneously target VEGF-A, a vascular growth factor that is expressed by various cellular components of the tumor microenvironment and the previously validated cancer target HER2. A HER2/VEGF-A TanCAR molecule would have wide applicability, because these targets are expressed on various tumors (HER2 is expressed in breast cancer, ovarian cancer, brain cancer, sarcomas and lung cancer; VEGF-A is targeted in brain, lung and colorectal cancer) and one can generate TanCAR T cells on a platform that, while personalized, is broadly applicable to various patient tissue types. In certain embodiments of the invention, combination therapy is employed with the invention, wherein other cancer treatments are provided to the individuals receiving the TanCAR therapy. For example, one can also target HER2 with trastuzumab (Herceptin®) and small molecule tyrosine kinase inhibitors and VEGF-A targeting agents, namely becizumab (Avastin®) and other cell therapy products and vaccines. The present invention also provides an advantage for the proposed product over conventional adjuvant agents given that the increasingly aging population has demanded a substantial shift towards low toxicity, targeted, QOL-favorable agents.

The duality of the approach (targeting both the tumor and the tumor complex, for example) is a major advantage over vaccines (such as Provenge®, the only FDA-approved immunotherapeutic approach against cancer, a first generation cell-based anticancer therapeutic that only targets a single antigen displayed on the surface of prostate tumor cells) as well as other targeted agents including for Herceptin® and Avastin®.

### I. Chimeric Antigen Receptors

Genetic engineering of human T lymphocytes to express tumor-directed chimeric antigen receptors (CAR) can produce antitumor effector cells that bypass tumor immune escape mechanisms that are due to abnormalities in protein-antigen processing and presentation. Moreover, these transgenic receptors can be directed to tumor-associated antigens that are not protein-derived. In certain embodiments of the invention there are CTLs that are modified to comprise at least a CAR, and in particular embodiments of the invention a single CAR targets two or more antigens.

In particular cases, the cytotoxic T lymphocytes (CTLs) include a receptor that is chimeric, non-natural and engineered at least in part by the hand of man. In particular cases, the engineered chimeric antigen receptor (CAR) has one, two, three, four, or more components, and in some embodiments the one or more components facilitate targeting or binding of the T lymphocyte to one or more tumor antigen-comprising cancer cells. In specific embodiments, the CAR comprises an antibody for the tumor antigen, part or all of a cytoplasmic signaling domain, and/or part or all of one or more co-stimulatory molecules, for example endodomains of co-stimulatory molecules. In specific embodiments, the antibody is a single-chain variable fragment (scFv). In certain aspects the antibody is directed at multiple target antigens on the cell surface of cancer cells, for example, although in some cases the target antigen is a secreted molecule from a cell and is not membrane-bound. In certain embodiments, a cytoplasmic signaling domain, such as those derived from the T cell receptor ζ-chain, is employed as at least part of the chimeric receptor in order to produce stimulatory signals for T lymphocyte proliferation and effector function following engagement of the chimeric receptor with the target antigen. Examples would include, but are not limited to, endodomains from co-stimulatory molecules such as CD28, 4-1BB, and OX40 or the signaling components of cytokine receptors such as IL7 and IL15. In particular embodiments, co-stimulatory molecules are employed to enhance the activation, proliferation, and cytotoxicity of T cells produced by the CAR after antigen engagement. In specific embodiments, the co-stimulatory molecules are CD28, OX40, and 4-1BB and cytokine and the cytokine receptors are IL7 and IL15.

The CAR may be first generation, second generation, or third generation (CAR in which signaling is provided by CD3ζ together with co-stimulation provided by CD28 and a tumor necrosis factor receptor (TNFr), such as 4-1BB or OX40), for example. The CAR may be specific for HER2, CD19, IL13R-alpha2, Tem8, FAP, EphA2 and/or VEGF-A although in some cases the CAR is specific for CD19, CD₂O, CD22, k light chain, CD30, CD33, CD123, CD38, ROR1, ErbB2, ErbB3/4, EGFr vIII, carcinoembryonic antigen, EGP2, EGP40, mesothelin, TAG72, PSMA, NKG2D ligands, B7-H6, IL-13 receptor α2, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, ALCAM, CD6, and/or CD44v6, for example.

In particular cases the CAR is specific for HER2, CD19, IL13R-alpha2, Tem8, FAP, EphA2 and/or VEGF-A, and in certain embodiments, the present invention provides chimeric T cells specific for HER2, CD19, IL13R-alpha2, Tem8, FAP, EphA2 and/or VEGF-A by joining an extracellular antigen-binding domain derived from the HER2-, CD19-, IL13R-alpha2-, Tem8-, FAP-, EphA2- and/or VEGF-A-specific antibody to cytoplasmic signaling domains derived from the T-cell receptor ζ-chain, with the endodomains of the exemplary costimulatory molecules CD28 and OX40, for example. This CAR is expressed in human cells, such as T cells, NK cells, or NKT cells, and the targeting of HER2-, CD19-, IL13R-alpha2-, Tem8-, FAP-, EphA2- and/or VEGF-A-positive cancers is encompassed in the invention.

### II. Cells

Embodiments of the invention include cells that express a CAR that targets two or more tumor antigens. The cell may be of any kind, including an immune cell capable of expressing the CAR for cancer therapy or a cell, such as a bacterial cell, that harbors an expression vector that encodes the CAR. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T cell (also known as TC, Cytotoxic T Lymphocyte, CTL, T-Killer cell, cytolytic T cell, CD8+ T-cells or killer T cell); NK cells and NKT cells are also encompassed in the invention.

In certain embodiments, it is contemplated that RNAs or proteinaceous sequences may be co expressed with other selected RNAs or proteinaceous sequences in the same cell, such as the same CTL. Co expression may be achieved by co transfecting the CTL with two or more distinct recombinant vectors. Alternatively, a single recombinant vector may be constructed to include multiple distinct coding regions for RNAs, which could then be expressed in CTLs transfected with the single vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the modified CTLs, where one wishes to terminate the treatment, the cells become neoplastic, in research where the absence of the cells after their presence is of interest, or other event. For this purpose one can provide for the expression of certain gene products in which one can kill the modified cells under controlled conditions, such as inducible suicide genes.

### III. Illustrative Exemplifications

By way of illustration, cancer patients or patients susceptible to cancer or suspected of having cancer may be treated as follows. CTLs modified as described herein may be administered to the patient and retained for extended periods of time. The individual may receive one or more administrations of the cells. In some embodiments, the genetically modified cells are encapsulated to inhibit immune recognition and placed at the site of the tumor. The cells may be injected at the tumor site or injected intravenously, for example.

In particular cases the individual is provided with therapeutic CTLs modified to comprise a CAR specific for two or more antigens. The cells may be delivered at the same time or at different times as another type of cancer therapy. The cells may be delivered in the same or separate formulations as another type of cancer therapy. The cells may be provided to the individual in separate delivery routes as another type of cancer therapy. The cells may be delivered by injection at a tumor site or intravenously or orally, for example. Routine delivery routes for such compositions are known in the art.

### IV. Introduction of Constructs into CTLs

Expression vectors that encode the CARs can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding at least the CAR and optionally another gene, or different DNA molecules having one or more genes. Other genes include genes that encode therapeutic molecules or suicide genes, for example. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

### V. Administration of Cells

The CTLs that have been modified with the construct(s) are then grown in culture under selective conditions and cells that are selected as having the construct may then be expanded and further analyzed, using, for example; the polymerase chain reaction for determining the presence of the construct in the host cells. Once the modified host cells have been identified, they may then be used as planned, e.g. expanded in culture or introduced into a host organism.

Depending upon the nature of the cells, the cells may be introduced into a host organism, e.g. a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described above at least in part.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

### VI. Nucleic Acid-Based Expression Systems

The bispecific TanCARs or multispecific TanCARs of the present invention may be expressed from an expression vector. Recombinant techniques to generate such expression vectors are well known in the art.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

B. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5 prime′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages, and these may be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

C. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™ 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

D. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention may be a viral vector that encodes one or more CARs of the invention. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

E. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

F. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

G. Vector Delivery and Cell Transformation

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

H. Ex Vivo Transformation

Methods for transfecting eukaryotic cells and tissues removed from an organism in an ex vivo setting are known to those of skill in the art. Thus, it is contemplated that cells or tissues may be removed and transfected ex vivo using nucleic acids of the present invention. In particular aspects, the transplanted cells or tissues may be placed into an organism. In preferred facets, a nucleic acid is expressed in the transplanted cells.

### VII. Kits of the Invention

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly ueful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments of the invention, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR as described herein and/or regulatory elements therefor.

In particular embodiments, there are one or more apparatuses in the kit suitable for extracting one or more samples from an individual. The apparatus may be a syringe, scalpel, and so forth.

In some cases of the invention, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### VIII. Combination Therapy

In certain embodiments of the invention, methods of the present invention for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with other therapies. In the context of the present invention, it is contemplated that cell therapy could be used similarly in conjunction with chemotherapeutic, radiotherapeutic, or immunotherapeutic intervention, as well as pro-apoptotic or cell cycle regulating agents.

Alternatively, the present inventive therapy may precede or follow the other agent treatment by intervals ranging from minutes to weeks. In embodiments where the other agent and present invention are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, present invention is “A” and the secondary agent, such as radio- or chemotherapy, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation based treatments. Combination chemotherapies include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing and also combinations thereof.

In specific embodiments, chemotherapy for the individual is employed in conjunction with the invention, for example before, during and/or after administration of the invention.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Embodiments of the Invention

Adoptive immunotherapy using activated and/or expanded CMV- or EBV-specific cytotoxic T lymphocytes (CTLs) has been successful in preventing malignant diseases associated with these viruses (Riddell et al., 1992; Walter et al., 1995; Rooney et al., 1998; Heslop et al., 1996). EBV-specific CTLs also had antitumor effects: none of the patient who received CTLs prophylactically developed lymphoma, in contrast to 11.5% of the controls (Rooney et al., 1998). Further, 11 of 13 patients who received CTLs as treatment for overt lymphoma achieved complete remissions (Gottschalk et al., 2005; Pakakasama et al., 2004; Gottschalk et al., 2001). The use of autologous EBV-specific CTLs has also been evaluated for patients with EBV-positive lymphomas and nasopharyngeal cancer with encouraging results (Straathof et al., 2005; Louis et al., 2008; Bollard et al., 2004). Beyond EBV-specific CTLs, autologous ex vivo expanded tumor infiltrating lymphocytes or T-cell clones to melanoma patients have also produced significant antitumor effects (Yee et al., 2002; Dudley et al., 2005; udley et al., 2008; Rosenberg et al., 2008; Hunder et al., 2008). These results indicate the usability of banked off-the-shelf antigen-specific T cells for the treatment of cancer and infectious disease and the feasibility of its commercialization.

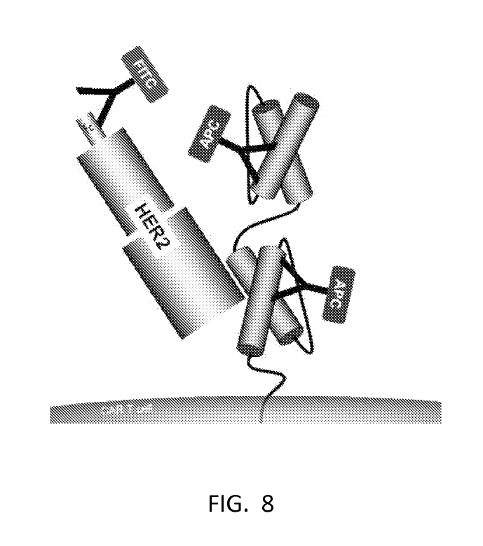

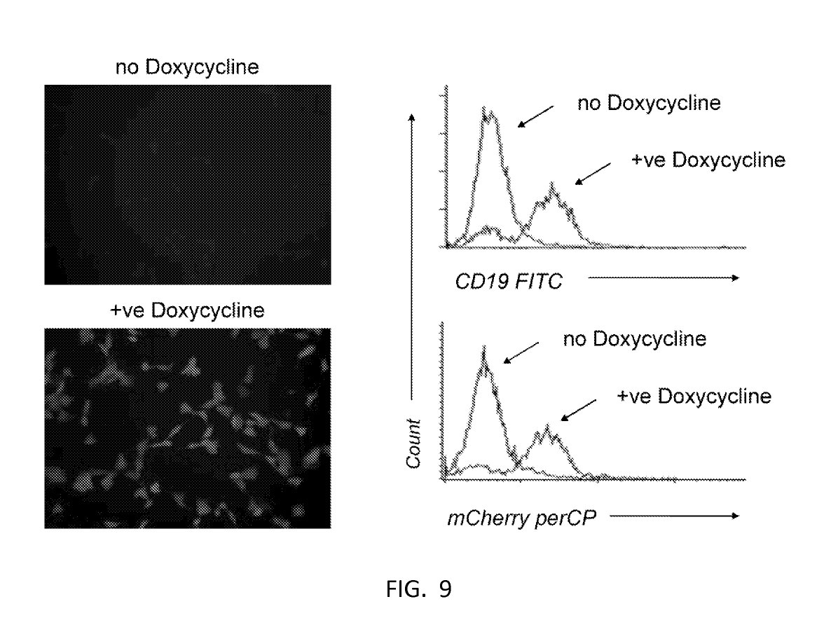

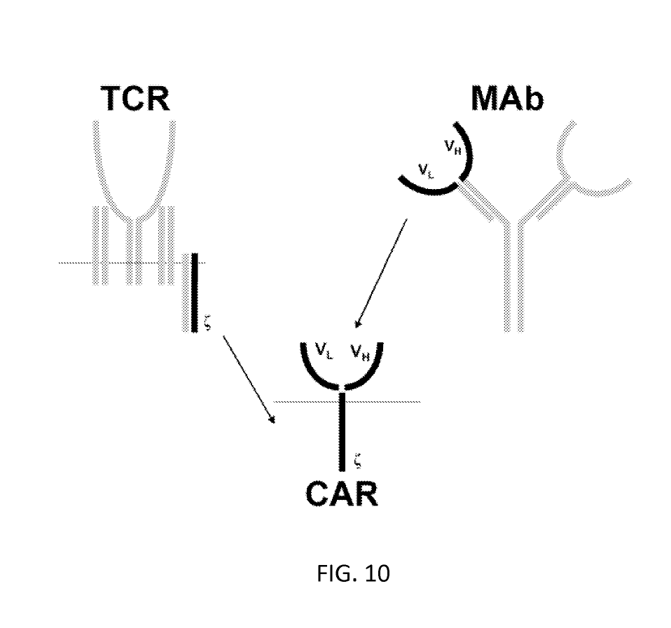

Genetically Engineered T cells: cell therapy by design. The broader use of antigen-specific CTLs for tumor therapy is currently limited by several factors, including 1) the reliable generation of tumor-specific T cells, 2) decreased MHC class I expression on tumor cells or defects in the antigen-processing machinery, 3) the presence of inhibitory T cells such as Th2 cells and/or Treg, at the tumor site, and 4) limited in vivo expansion of adoptively transferred T cells. One strategy to overcome many of these limitations is the genetic modification of T cells to express chimeric antigen receptors (CARs; FIG. 10). CARs provide T-cell activation in a non-MHC-restricted manner and therefore circumvent some of the major mechanisms by which tumors avoid MHC-restricted T-cell recognition, such as downregulation of HLA class I molecules and defects in antigen processing. Moreover, expressing CARs with multiple signaling domains in T cells renders them resistant to the inhibitory effects of regulatory Tregs (Loskog et al., 2004). Lastly, CAR expressing T cells can be readily prepared in large quantities ex vivo for clinical applications. Indeed, preclinical data targeting HER2 are currently translated into FDA and IRB approved clinical trials for Glioblastoma (NCT01109095; PI: Ahmed), sarcomas (NCT00902044; PI: Ahmed) and lung cancer (NCT00889954; PI: Gottschalk). The studies so far have shown that systemic infusion of HER-specific CAR T cells is safe. Several other studies (Pule et al., 2008; Savoldo et al., 2011) and from others have indicated that infusion of CAR T cells is safe and is associated with clinical benefits (Pule et al., 2008; Morgan et al., 2006; Till et al., 2008; Park et al., 2007; Porter et al., 2011).

The Tumor Microenvironment: targeting the tumor complex. It is now becoming evident that cancers are heterogenous cellular complexes whose growth is dependent upon reciprocal interactions between the genetically initiated “cancer cells” and the dynamic microenvironment in which they induce (Tisty and Coussens, 2006). These cellular elements include the tumor endothelium, epithelial cells as well as elements from the immune system (Bissell et al., 2005). This crosstalk between the “cancer” cell and cellular elements of its microenvironment is largely mediated by soluble factors. One such factor is, the proangiogenic polypeptide vascular endothelial growth factor (VEGF)-A (Esposito et al., 2004; Barbera-Guillem et al., 2002). VEGF-A is highly expressed in developing tumors and is secreted by various cellular elements of the tumor microenvironment, including: the tumor endothelial cells, epithelial cells as well as inflammatory and immune infiltrates (Bergers et al., 1998; Bergers et al., 2000). However, the bioavailability of VEGF-A is limited, as it is tethered to secreting cells via membrane-spanning or anchorage domains until cleaved by extracellular proteinases (mostly MMP-2 and MMP-9) (Bergers et al., 1998; Bergers et al., 2000; Egeblad et al., 2010). Uncleaved VEGF-A thus represents a versatile marker for a wide array of cellular elements in the tumor microenvironment that could effectively opsonize these cells for VEGF-A-specific T cells. Very importantly, apart from wound healing areas, the expression of VEGF-A is negligible in normal tissues (Baillie et al., 2001; Hanrahan et al., 2003).

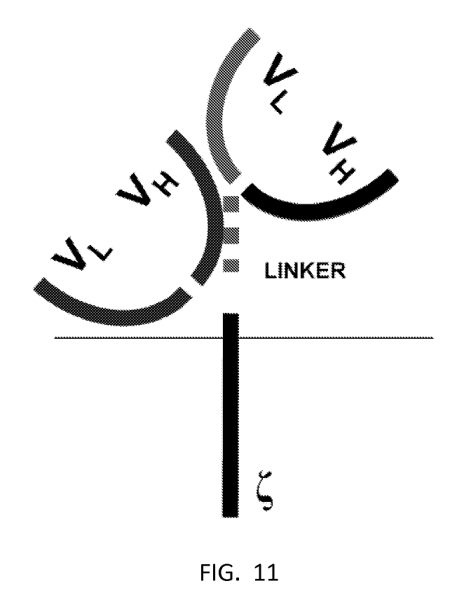

Tandem Chimeric Antigen Receptor. Rendering an individual T cell bispecific is useful in offsetting tumor escape, improving T cell activation and most importantly in simultaneously targeting the tumor and elements from its microenvironment (FIG. 11).

Bispecific T lymphocytes expressing this tandem CAR molecule (TanCAR) distinctly and specifically recognized and killed tumor cell targets positive for either molecule. Furthermore, an inducible system is used to study the TanCAR functionality in the presence of single and dual target molecules on tumor cells. TanCAR T cells exerted synergistic functionality upon simultaneous engagement of both target molecules on the same cell. Moreover, they maintained their effector function despite downregulation of one target molecule, a characteristic that would maintain cytotoxicity againsT antigen escape tumor variants.

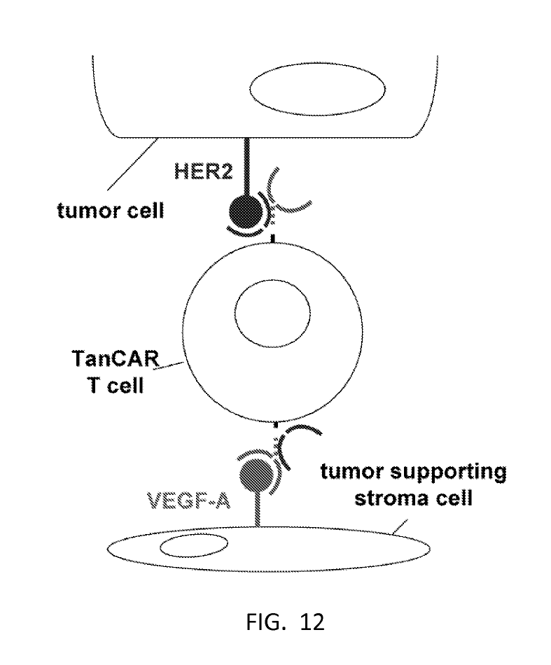

Embodiments of the present invention show how to systematically design, build and functionally test a TanCAR molecule that will target HER2, a validated tumor “cell” target, and VEGF-A, a molecule that marks various cellular elements of the tumor “microenvironment” (as illustrations of the invention only). When grafted on effector T cells, this bispecific cellular product could broadly target the tumor cell and the supporting cellular elements (FIG. 12).

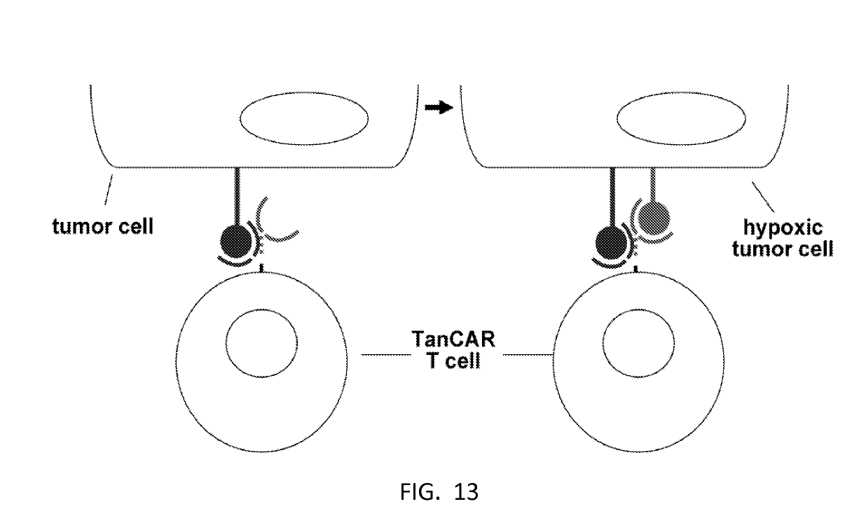

By virtue of the metabolic activity of cancer cells that is disproportionate to the vascular supply and venous drainage, tumors often have a hypoxic milieu. During hypoxia, tumor cells secrete VEGF-A (Baillie et al., 2001; Hanrahan et al., 2003; Currie et al., 2004). Bispecific TanCAR T cells could thus engage both HER2 and VEGF-A simultaneously (FIG. 13). This was shown in the exemplary TanCAR described herein to synergistically activate T cells.

While it is currently evident that there is a dire need for novel biologically-based strategies to achieve control of currently incurable malignancies, in specific embodiments of the invention the need is addressed to adopt broad (even multimodality) approaches that undermine the whole tumor “complex”. Two versatile cancer antigens: HER2, a well-established target in multiple carcinomas, sarcomas and tumors of the neuraxis, and VEGF-A, an emerging target for multiple common malignancies, may be employed as examples. This makes the domain of applicability of the cellular product quite broad. In embodiments of the invention, one can generate a master cell bank of closely matched (matching at as low as 1-2 antigens) off-the-shelf product by securing 25 lines (for example) covering the most common HLA type, and this will make this therapeutic option available for >85% of patients of diverse ethnic backgrounds.

### Specific Embodiments

In specific embodiments of the invention: a) computational platforms predict the optimum structure (s) for an exemplary HER2/VEGF-A bispecific TanCAR molecule that will simultaneously dock to both target molecules, b) bispecific TanCAR T cells exert distinct functionality against either antigen alone as well as enhanced functionality against both antigens simultaneously, and C) that HER2/VEGF-A TanCAR T cells exhibit enhanced antitumor activity against established tumor xenografts compared to T cells targeting HER2 or VEGF-A alone or a pooled product thereof.

Modeling and Construction of a HER2/VEGF-A Bispecific TanCAR Molecule. One can use computational platforms to design the most favorable TanCAR models that will dock to both HER2 and VEGF-A (as examples only) simultaneously with the lowest energy conformations predicting the highest avidity to both targets. Thereafter, one can synthesize up to ten (for example) most optimal TanCARs using chemical synthesis and conventional cloning methodologies in clinically translatable retroviral vectors.

Functional Testing of the HER2/VEGF-A Bispecific TanCAR T cells. One can use standard immunoassays to test the in vitro functionality of TanCAR T cells against HER2 and VEGF-A expressing tumor cells and cellular elements from the tumor microenvironment, respectively. Furthermore, one can use a hypoxia culture system to induce VEGF-A in tumor cells and test the effect of co-targeting both molecules on T cell activation. The in vivo efficacy of HER2/VEGF-A TanCAR T cells can be tested in an orthotopic murine model of Glioblastoma against T cells targeting HER2 or VEGF-A alone or a pooled product thereof.

### Modeling and Construction of a HER2/VEGF-A Bispecific TanCAR Molecule

Generating a Computational Model of Favorable Simultaneous Docking of HER2 and VEGF-A Individually and Simultaneously.

(a) Rationale. Spontaneous folding of amino acid chains into highly organized, biologically functional three-dimensional protein structures, such as a CAR, can be a challenge to the design of novel protein molecules. In particular; protein misfolding, mispairing and malfunction or dysfunction, have traditionally impeded attempts at production of molecules with multiple specificity. Recent advances in computational platforms, through characterization of the underlying energy landscapes as well as the dynamics of the polypeptide chains in all stages of the folding process, made the structure prediction, analysis, and design of a novel protein molecule, such as a tandem CAR, more feasible. Furthermore, docking platforms have recently made it possible to predict, with high-accuracy, the docking between candidate molecules in a manner that could correlate well with their functionality.

(b) Experimental Design. Computational platforms (ModWeb®, RosettaDock® and Firedock®) are used to study the most favorable models for docking of both scFv's, the order of these scFv's and the length of the linker molecule separating them as well as other permutations that will yield the lowest energy for docking and predict the highest avidity of the TanCAR molecule to both targets simultaneously.

(i) One can consider the most favorable provisional global TanCAR structure, such as whether or not it is proximal to distal arrangement of scFv's. One can consider the most favorable linker length. Models for the bispecific HER2/VEGF-A TanCAR are constructed using ModWeb®, an automated web server for protein structure modeling. The full length TanCAR sequence is submitted to the ModWeb server, which will identify the two antibody-like fragment domains. From these templates, a homology model is constructed spanning residues of high potential for binding, but also including various lengths of the 10-20 amino acid long GLY-SER linker separating the two variable antigen recognition domains. The structure for the HER2 target is available (PDB ID: 1N8Z), while a homologous structure for VEGF-A has been identified using the BioInfoBank® metaserver (DB00112). A model for pertinent residues of VEGF-A is constructed with Modeller® V9.1 using 3MJG (12.59% sequence identity) as a structural template. Initially, pairwise docking is performed with PatchDock® using the individual TanCAR domains and the corresponding receptor; residues 39-155 (known from prototype TanCAR work) corresponded to the HER2 binding portion of TanCAR, while residues for VEGF-A binding portion of the TanCAR can be researched.

(ii) One can consider the most favorable model of docking of HER2 and VEGF-A to their respective scFv's. Fits are evaluated visually and based on their PatchDock® score. For the CAR-HER2 docking, results are additionally filtered based on previous studies that had suggested binding residues and the prototype TanCAR work. Further refinement of the individual candidate dockings are done using FireDock®. Candidate dockings from both TanCAR-HER2 and TanCAR-VEGF-A are then combined in UCSF Chimera® and evaluated for steric clashes.

(iii) One can consider which assembled TanCAR molecule designs yield the most favorable energy docking to both targets simultaneously. The final model for the HER2-TanCAR-VEGF-A docking is selected based on lowest global energy in each of the pairwise dockings from FireDock® an steric constraints in the entire assembly, in specific embodiments. One can identify up to 10 molecular designs (for example) with favorable profiles.

One can anticipate being able to identify 5-10 most favorable structures of the HER2/VEGF-A TanCAR for functional testing, because: a) the FRP5 docking to its HER2 binding domain is already solved; b) similarly, one can construct the 2nd svFv and determine the lowest energy docking to VEGF-A. Because of the respective lengths, FRP5 in the juxta-membrane position may be more relaxed and allow for simultaneous binding because the FRP5 molecules binds to the distal-most 4 loupes of HER2. Lastly, the linker may be 10-20 amino acid residues as established by others. If one cannot identify the favorable docking sites, one can use sites with the least energy. If VEGF-A/HER2 is similar to HER2/VEGF-A, one can test both functionally and use the better construct.

Construction of HER2/VEGF-A TanCAR Candidate Molecules for Functional Testing. The top ten candidate TanCAR molecules with the lowest energy conformations of simultaneous docking to both HER2 and VEGF-A are constructed using chemical gene synthesis and in-house cloning methodologies followed by sequence verification. Clinically translatable retroviral vectors (Moloney Murine Leukemia Virus; MoMuLV-based) are used to allow for future generation of a clinical grade vector.

Computational Platforms can efficiently guide the TanCAR design predicting a high avidity of the proposed molecules to the respective targets. Other factors may play a role, such as protein folding, whether complete surface expression will occur and if mis-pairing of molecules will occur. Force expression and expression analysis may be utilized to verify the engraftment of the TanCAR on T cells.

Exemplary Experimental Design

(i) Extracellular Domain Transgene Optimization, Synthesis and Verification: The designed transgene DNA sequence is modified to include restriction enzyme sites at the cloning sites and exclude any inadvertently inserted sites within the translation elements then optimized using the GeneOptimizer® software for maximum protein production. The extracellular domain is synthesized by by GeneArt® Inc. using oligonucleotides then cloned into the Gateway® entry vector pDONR™ 221, standard cloning vector then sequence-verified.

(ii) Cloning of the TanCAR Transgene into a MoMuLV Retroviral Construct. This antigen recognition domain is subcloned in frame into a SFG retroviral vector containing a short hinge, and the transmembrane and signaling domain of the costimulatory molecule, CD28 and the signaling domain of the T-cell receptor ζ-chain (Moritz et al., 1994; Rossig et al., 2001; Pule et al., 2005).

(iii) Verification of the Structure of the Retroviral Constructs. The structure of the whole TanCAR constructs are confirmed using restriction digests. The 5′-and 3′ as well as the 3′-5′ sequence of the TanCAR molecules are confirmed using single base pair pyro-sequencing.

### Functional Testing of the HER2/VEGF-A Bispecific TanCAR T Cells

(1) In Vitro Immunological Testing.

(a) Standard immunoassays (coculture assays, proliferation, cytokine release using CBA arrays, ELISpot and ELISA and cytotoxicity assays) are used to test the in vitro functionality of TanCAR T cells, grafted with the various candidate TanCAR molecules, against HER2 and VEGF-A expressing tumor cells and cellular elements from the tumor microenvironment, respectively. A hypoxia culture system is used to induce VEGF-A secretion in tumor cells and test the effect of co-targeting both molecules on T cell activation and their effector functions. Furthermore, the best functioning TanCAR T cell line is tested against CAR T cells targeting only HER2 or VEGF-A individually in the same system.

(b) Experimental Design.

(i) Blood donors and target GBM tumor and stroma cell lines. Blood samples are obtained from healthy donors consented on a protocol approved by the IRB of Baylor College of Medicine to generate effector cells. The GBM line U373 and U87 and the brain tumor endothelial cell line HBMEC (ScienCell Inc.) may be used to test the in vitro functionality of these effectors. MDA-MB-468, the breast cancer cell line, can serve as the negative control because it lacks both targets.

(ii) Retrovirus production and transduction of T cells. To produce retroviral supernatant, 293T cells are cotransfected with retroviral vector containing plasmid for each candidate TanCAR, Peg-Pam-e plasmid encoding the sequence for MoMLV gag-pol, and plasmid pMEVSVg containing the sequence for VSV-G, using GeneJuice® 39; 40. Transient supernatants containing the retrovirus are collected 48 and 72 hours later. OKT3 activated T cells are transduced with retroviral vectors as described (Straathof et al., 2005; Dotti et al., 2005). Briefly, peripheral blood mononuclear cells (PBMC) are isolated by Lymphoprep gradient centrifugation. 5×10⁵ PBMC per well of a 24-well plate were activated with OKT3 at a final concentration of 1 μg/mL. On day 2, IL-2 is added at a final concentration of 50 units/mL, and on day 3 cells are harvested for retroviral transduction. One can precoat non-tissue culture treated 24-well plates with fibronectin. Subsequently, 2.5×10⁵ T cells per well are transduced with retrovirus in IL-2. After 48-72 hours cells are removed and expanded in IL-2 per mL for 10-15 days after TanCAR surface expression by flowcytometry is verified using a) a FAB specific antibody and b) an FRP5 (HER2 scFv) recognizing strategy using HER2.Fc followed by anti-Fc Ab.

(iii) Do TanCAR-expressing T cells distinctly recognize HER2 and VEGF-A, individually? We will use the previously described retroviral transduction system to force-express the candidate TanCAR molecules on a number of normal donor T cell blasts (n=3 at least) to produce HER2/VEGF-A Tan CAR T cell lines. IFNγ release and cytotoxicity assays are used to test the specific recognition and lysis of individual target molecules against cells that individually express HER2 or VEGF-A.

(iv) Does the simultaneous recognition of VEGF-A (in addition to HER2) on hypoxic tumor cells result in improved antitumor activity of TanCAR T cells? While tumor cells do not secrete VEGF-A under normoxic conditions, VEGF-A secretion is induced by hypoxia in an attempt to induce vasculogenesis and reverse the hypoxia and interstitial acidity, both of which are detrimental to tumor growth. Similar to cells from the tumor microenvironment, secreted VEGF-A remains tethered to the tumor cell membrane until enzymatically cleaved, and is amenable to surface detection. This scenario is favorable for TanCAR T cell activation since co-expression of a second CAR target (as shown by initial studies) results in enhanced T cell activation and target cell killing. One can preincubate tumor cells in hypoxia chambers, verify the induction of VEGF-A on the tumor cell surface and perform a set of co-culture experiments, cytokine analysis and cytotoxicity assays to test for this possibility.

(v) Which is the most favorable TanCAR molecule design that confers the best functionality on recipient primary T cells? In a separate set of studies, the candidate TanCAR cell lines are tested, in parallel, for their differential effector functionality against U373 and U87 and HBMEC cells as well as on hypoxic tumor cells. Collective data is compiled and tested statistically to determine which TanCAR confers the most favorable profile on T cell activation and functionality in vitro against both individual target molecules and simultaneously against both. From the most favorably functioning TanCAR T cell line, one can produce a master cell bank for further in vivo testing and testing in the animal model from at least three healthy donors.

Based on studies using the prototype HER2/CD19 TanCAR, one can anticipate that the most TanCAR designs are secreted on the cell surface and distinctly recognize both HER2 and VEGF-A. In the event that there is no TanCAR expression, one can change the leader (secretion) sequence on the construct, for example. In the event that the TanCAR T cells fail to recognize either or both targets, one can synthesize and screen the next set of models and/or change the epitope on HER2 or VEGF-A, for example. One can anticipate that the killing of hypoxic GBM cells is enhanced because of the expression of VEGF-A. In the event that this does not occur, one can introduce a hypoxia-inducible element to enhance TanCAR expression in hypoxic conditions.

(2) Exemplary Rationale.

Because the simulation of the steric orientation of the tumor cell to tumor stroma is quite limited in in vitro systems, one can use an animal model to test the functionality of HER2/VEGF-A Tan CAR T cells. The expression has been validated, and preclinical targeting of HER2 in models of GBM and a HER2-targeting adoptive immunotherapy trial is currently ongoing at the CAGT (HERT-GBM; NCT01109095; PI: Ahmed). Similarly, multiple groups have extensively characterized VEGF-A expression both in GBM models and in primary human tumors. Indeed, bevacizumab (Avastin® has become the main salvage line for adult GBM). For these reasons, one can test the in vivo efficacy of HER2/VEGF-A TanCAR T cells in an orthotopic murine model of GBM, a model which combines both targets. One can address considerations pertaining to the advantage of the TanCAR approach over targeting either HER2 or VEGF-A, over combining these products or over bispecific T cell co-expressing both HER2 and VEGF-A CARs.

Experimental Design.

(i) The GBM orthotopic xenograft animal model (Ahmed et al., 2010). VEGF-A is largely conserved in human and mouse and contribution from the tumor bed serves as a target, in specific embodiments. Furthermore, established tumor xenografts can have a hypoxic center serving as an adequate model for target coexperession. Recipient NOD-SCID mice are anesthetized, head shaved, then immobilized in a Cunningham™ stereotactic apparatus. The tip of a 31G ½ inch needle is introduced to coordinates corresponding to the right frontal cortex. Firefly-luciferase expressing primary GBM cells and U373 GBM cell line is injected. All animals get progressively growing xenografts as evidenced by bioluminescence imaging. Mice are then randomly assigned to one of the experimental groups. After tumor establishment, effector T cells are injected into the same stereo-coordinates. The mice are bioluminescence imaged thrice weekly to monitor tumor progression. Pathological examination are performed on tumor explants. For in vivo testing, one can utilize imageable eGFP.Firefly luciferase expressing U373 cells, for example.

(ii) Do HER2.VEGF-A TanCAR T cells induce better tumor control and survival over targeting either molecule individually using HER2CAR or VEGF-A CAR T cells? Tumors are established in animals as outlined above and animals are randomized to receive HER2.VEGF-A TanCAR T cells, HER2CAR T cells or VEGF-A CAR T cells. Tumor volumes are assessed using bioluminescence and survival analysis is performed at day 90. Control groups receive PBS or non-transduced T cells from the same donor. Statistical analysis is performed as is a Kaplan-Meier plot to assess survival differences. All tumor explants are assessed pathologically for tumor antigen expression and survival of T cells.

(iii) Is co-targeting HER2 and VEGF-A using HER2.VEGF-A TanCAR T cells better than a pooled product HER2CAR or VEGF-A CAR T cells, or Bi-specific T cell products? One can test how a pooled product of two cell lines (HER2CAR T cells plus VEGF-A CAR T cells) or a product of T cells co-expressing both HER2CAR and VEGF-A CAR compares to HER2/VEGF-A TanCAR T cells in the ability to induce tumor regression and confer a survival advantage on treated animals.

By virtue of identifying two target molecules simultaneously, TanCAR T cells achieve better tumor control and confer a better survival advantage over targeting single molecules. Further, by docking to two targets simultaneously, enhanced T cell activation gives them an advantage over a pooled product targeting both antigens individually. This is quite advantageous in such a scenario where antigen expression is modest, as in HER2 on GBM cells. Lastly, while co-grafting multiple CAR molecules on T cells results in enhanced activation; yet by its ability to collapse the tumor complex, TanCAR T cells will achieve better tumor control. The inventors have constructed other clinically relevant TanCAR molecules to combine multiple tumor targets (IL13Rγ2 or EphA2) that may be utilized, in certain embodiments.

### Example 2

### A Chimeric Antigen Receptor Molecule Mediates Bispecific Activation and Targeting of T Lymphocytes

BACKGROUND: The downregulation or mutation of target antigens is a common tactic creating antigen loss escape variants. Targeting multiple antigens on tumor cells, simultaneously, is useful to offset this escape mechanism and possibly allow for simultaneous targeting of the tumor and elements of its microenvironment.

METHODS: The inventors used protein structure prediction and docking to construct a chimeric antigen receptor (CAR) molecule exodomain by joining two single chain variable fragments (scFv) molecules specific for CD19 and HER2 (as examples only). While CD19 and HER2 are not naturally coexpressed on normal or malignant mammalian cells, using them allowed the inventors to distinctly test the bispecific functionality of this CAR and its ability to activate T cells by binding to either or both target molecules. This exodomain was tethered to a hinge and transmembrane and signaling endodomain of the costimulatory molecule, CD28, and the C chain of T cell receptor. This bi-specific molecule was expressed on CD3/CD28 activated T cells by retroviral transduction. The functionality of C-CAR expressing bispecific T cells was tested in cytotoxicity and cytokine release assays.

RESULTS: Modeled structures and docking produced complexes with favorable interaction of the C-CAR and the published CD19 and HER2 sequences. The sequence of the C-CAR exodomain was confirmed using restriction enzyme digestion and single nucleotide sequencing. T cells expressed both the CD19 as well as the HER2 scFv as judged by FACS analysis. In cytotoxicity assays, C-CAR transduced T cells recognized and killed both CD19 as well as HER2 positive tumor cell targets. Soluble HER2 protein blocked tumor cell lysis in a HER2 protein-dependent manner. Similarly, CD 19-blocking antibodies inhibited the CD19 killing in an antibody concentration dependent manner. C-CAR grafted T cells secreted both IFN-γ and IL-2 in coculture with CD19 and HER2 positive tumor cells. CD19 negative, HER2 negative target cells were not lysed and induced no cytokine release.

CONCLUSION: This novel chimeric antigen receptor confers bispecific effector functions to T cells. T cells targeting two antigens simultaneously are useful to improve current T-cell therapy approaches for cancer by allowing for targeting of multiple antigens expressed by the tumor or its microenvironment.

### Example 3

### Requirements for a Bispecific “Tandem” Chimeric Antigen Receptor: TanCAR

Predictive molecular modeling was used to interrogate the hypothetical structure of a bispecific tandem CAR and provide a rational basis for designing a potentially optimized and functional molecule. The inventors developed an exemplary molecule, the TanCAR, to simultaneously target the B-cell antigen CD19 and the human epidermal growth factor receptor 2 (HER2/neu; also known as ErbB-2, CD340 and p185). The inventors considered that while CD19 and HER2 are not naturally co-expressed on normal or malignant cells, using them as targets would allow them to directly test the effects of binding one or both target antigens, and the consequences of such binding on T-cell activation and target cell killing. Furthermore, a crystal structure of HER2 was available. While the structure of CD19 is unknown, several potential structural templates, with relatively high sequence similarity, are available for constructing a homolog model of CD19. Coupled with a model on TanCAR, it is possible to reliably assess the docking potential of the these target molecules.

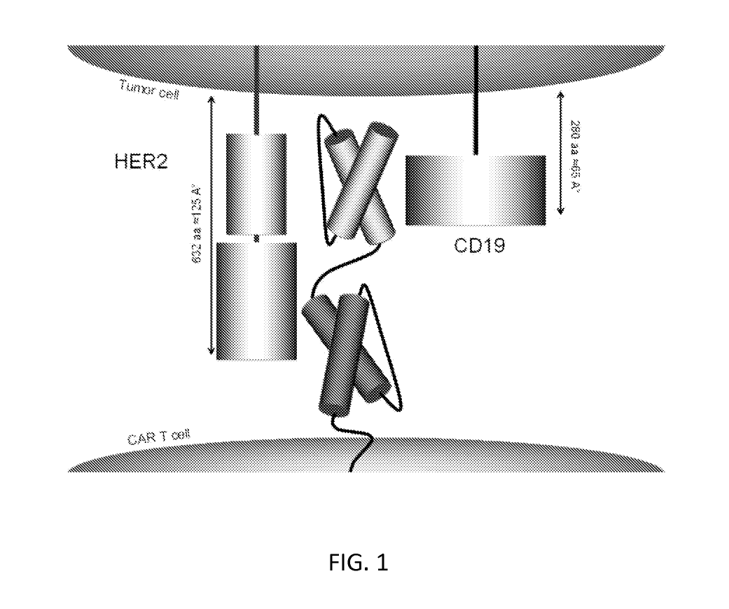

The extracellular domain of the TanCAR was designed to include a CD 19-specific scFv antibody fragment followed by a Gly-Ser linker, a HER2-specific scFv (FRP5) and another short Gly-Ser tandem repeat hinge (Marcotte et al., 1999; Matsushima et al., 2008). Gly-Ser tandem repeats represent a highly flexible non-cleavable structure that would allow for near-free motion of the TanCAR subunits. The intracytoplasmic domain of the exemplary TanCAR molecule consists of a CD28 signaling moiety followed by the T-cell receptor (CD3 complex)-chain, as previously described (FIG. 1) (Ahmed et al., 2010; Rainusso et al., 2011; Nakazawa et al., 2011; Ahmed et al., 2009).

### Example 4

### Computational Docking of the Proposed Tancar Design to Target Molecules

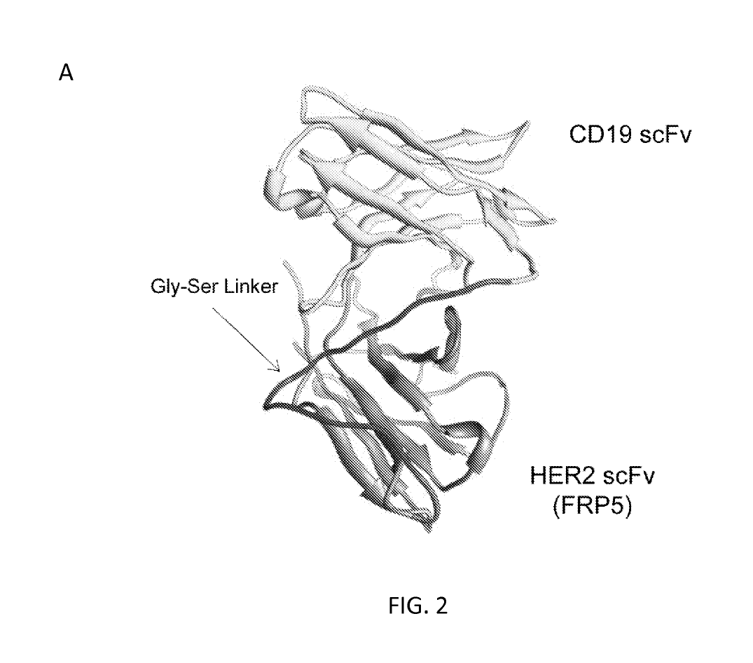

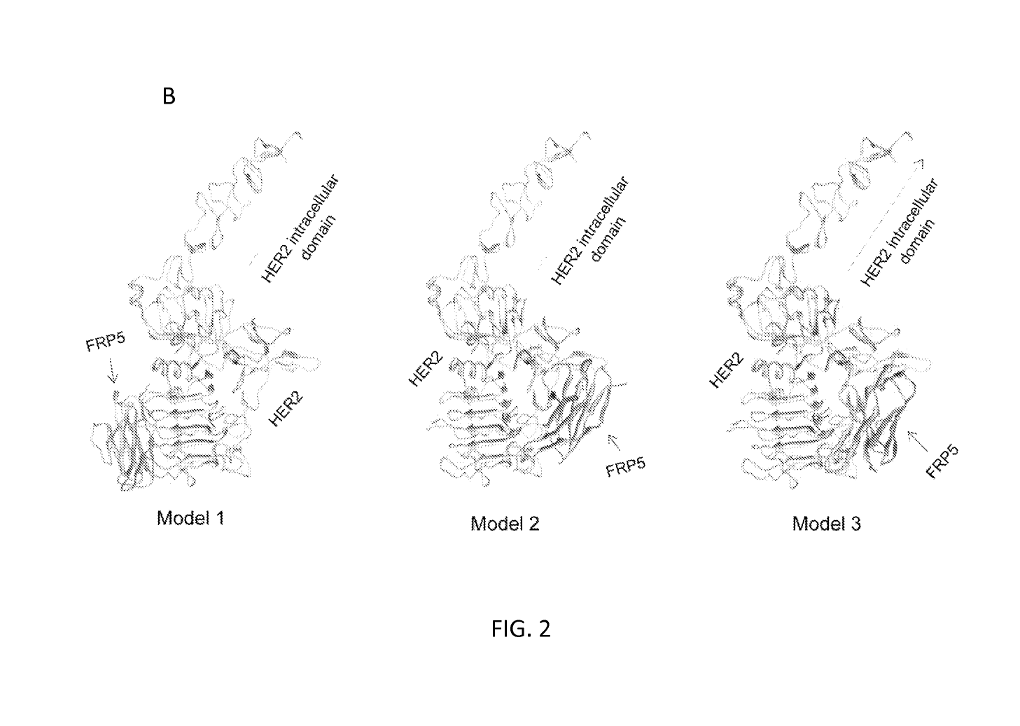

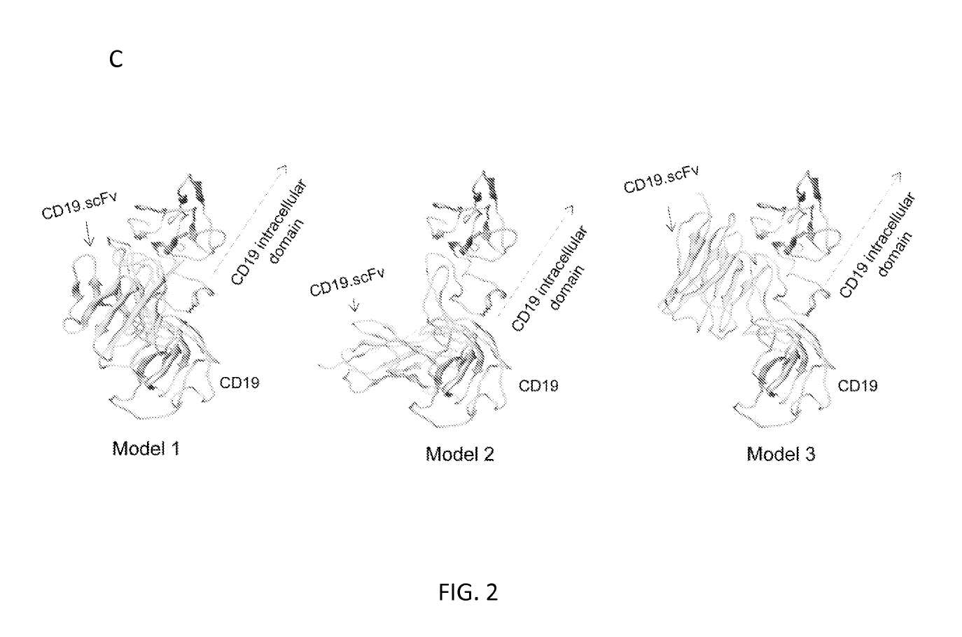

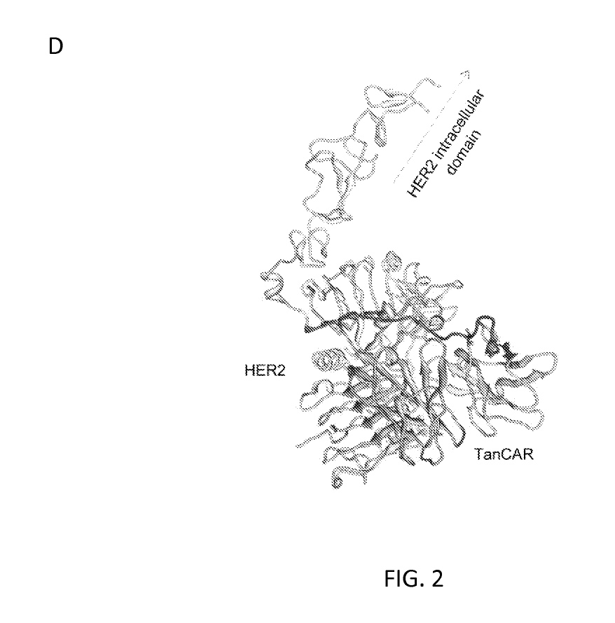

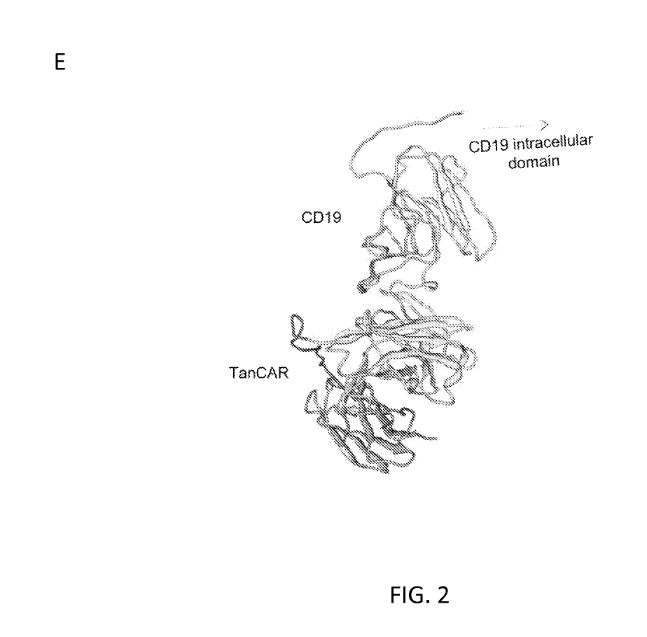

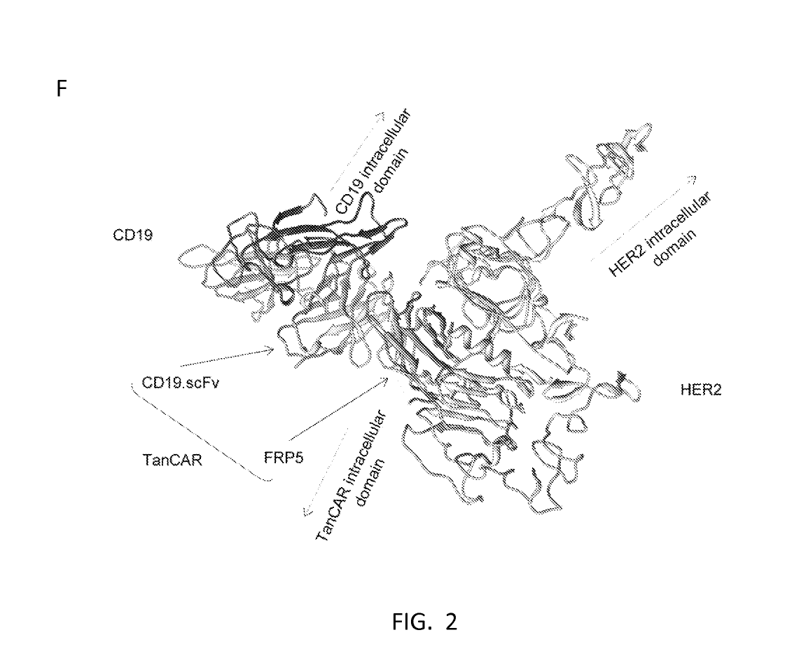

To characterize this exemplary molecular arrangement, the inventors generated a structural model of the TanCAR using the protein structure modeling webserver, ModWeb (Pieper et al., 2011; Eswar et al., 2003). Because of the respective lengths of HER2 (632-amino acids; 125 Å) and CD19 (280 amino acids; 65 Å) extracellular domains and the knowledge that FRP5 binds to the distal-most 4 loops of HER2 (W. Wels; unpublished data), the inventors placed HER2.scFv (FRP5) in the juxta-membrane position and the CD19.scFv in the distal position to allow for more relaxed and potentially simultaneous binding. Four structural templates with greater than 58% sequence identity were identified using ModWeb, from which a model for residues 39-285 of TanCAR was constructed. This model contained the two single chain antibody variable fragments (scFv) tethered to a hinge, transmembrane and signaling domains separated by a Gly-Ser linker (FIG. 2A).

Using the TanCAR model, the next step was to assess how it might interact with CD19 and HER2. While the structure of HER2 was known (Cho et al., 2003), no structure of CD19 is currently available. As such, a homology model for CD19 was generated using the 3MJG crystal structure as a structural template for residues 1-272 within the Modeller software (Shim et al., 2010).

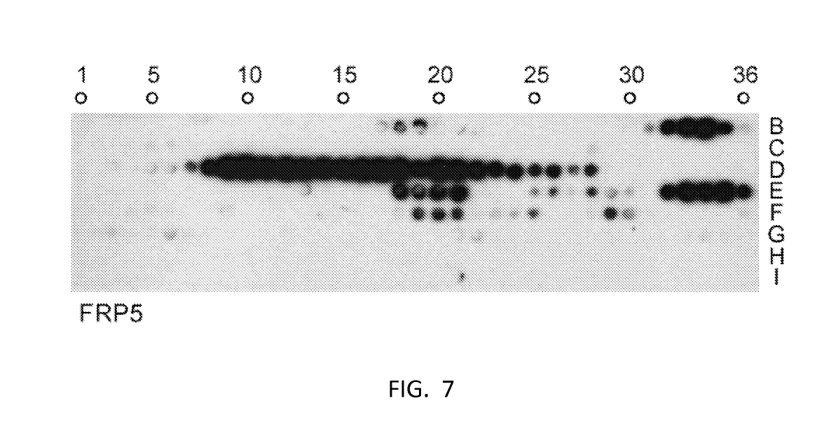

Using a combination of Patchdock and FireDock (Andrusier et al., 2007; Schneidman-Duhovny, et al., 2005), automated tools for docking and refining two structures based on shape complimentarity, the TanCAR model was docked pairwise to the HER2 and CD19 structures (FIGS. 2B, 2C). Results from the pairwise dockings were screened based on the overall score and agreement with known interaction sites from peptide spotting experiments in the case of HER2 (FIG. 7) (Gerstmayer et al., 1997).

The pairwise dockings were then visualized and aligned in UCSF's Chimera (Pettersen et al., 2004); the ensemble dockings were evaluated for global energy, agreement with interaction sites and steric clashes. This composite docking algorithm yielded only one docking combination that was sterically possible in which the HER2 and CD19 structures were bound to the TanCAR structure without any clashes (FIG. 2D-F).

Based on this docking, both the HER2 and CD19 protein structures are arranged such that their N-termini are essentially orientated in the same direction. Because the CD19 structure is approximately 50 percent of the size of HER2, there is a separation of ˜93 Å between the N-termini of both molecules. Differences in orientation of CD19 and HER2 along the cell membrane, as well as variations in the cell membrane itself, could account for the difference between the size and orientation of the receptors.

While the interface between TanCAR and HER2 is predominated by hydrophobic residues along beta sheets in both molecules, three potential salt bridges exist between Ser48:Arg135, Ser66:Arg56 and Arg12:Asp89 in TanCAR and HER2, respectively. Similarly, the interface between TanCAR and CD 19 is primarily characterized by hydrophobic residues in beta sheets of both structures, though more loops from CD19 appear to be involved. Again, three potential salt bridges stabilizing the scFv—target antigen interaction are possible between G1u221:Arg120, Arg232:Asp109 and Asp238:Arg244 in TanCAR and CD19, respectively. From the in silico docking of these molecules, it would appear that the potential interactions of TanCAR with the target molecules could accommodate the intended bi-specificity, and as such, the inventors used this arrangement as an initial model to explore the ability of TanCAR to interact with the target molecules.

### Example 5

### Construction, Delivery and Expression of the TanCAR Encoding Transgene

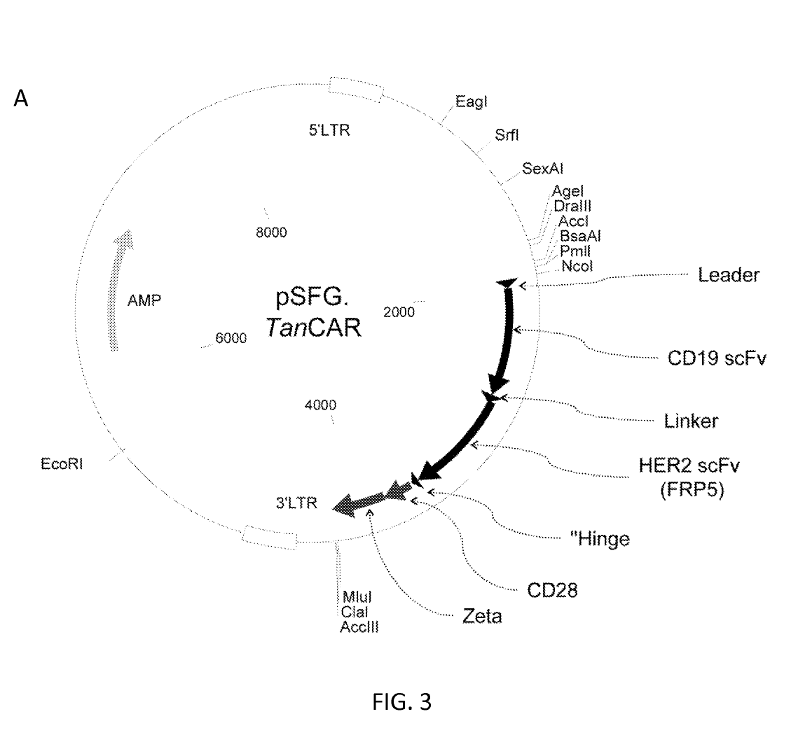

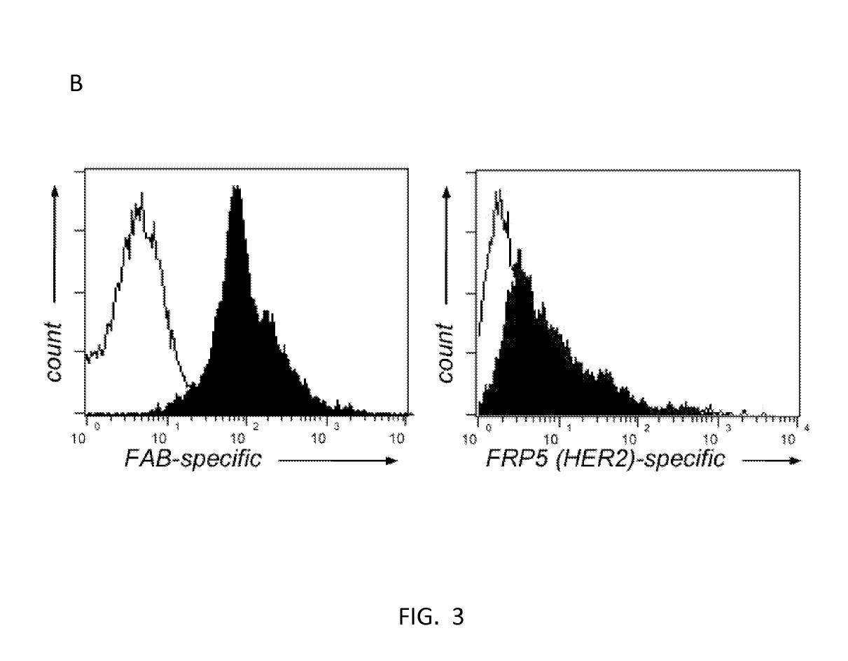

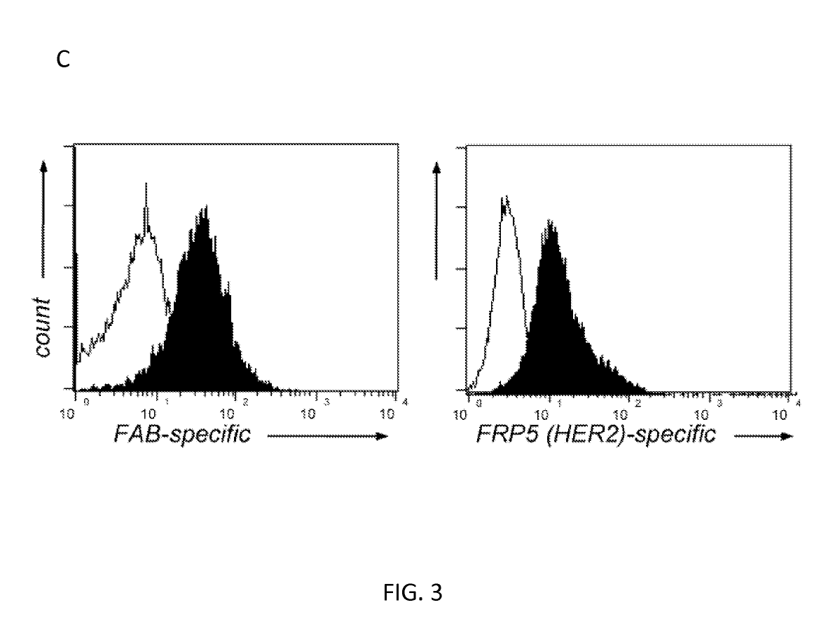

The modeled bispecific extracellular domain (excluding the Gly-Ser tandem repeat hinge), composed of the CD19 and HER2 scFv fragments in tandem and separated by a linker, was assembled on Clone Manager®, and modified to introduce the desired and remove unwanted restriction enzyme sites and optimized for maximum protein production using the GeneOptimizer® software (Raab et al., 2010). The in silico design of the TanCAR extracellular domain was synthesized as a DNA fragment by custom synthesis and then subcloned in frame into an SFG retroviral vector containing a short hinge, the transmembrane and signaling domain of the co-stimulatory molecule, CD28, and the signaling domain of the T-cell receptor (CD3 complex) ζ-chain (FIG. 3A) (Moritz et al., 1994; Rossig et al., 2001; Pule et al., 2005). The resulting TanCAR transgene was then expressed in human embryonic kidney (HEK) 293T cells as previously described (Ahmed et al., 2007). By flow cytometric analysis, the inventors determined approximately 89% and 59% of 293T cells as TanCAR positive using anti-Fab antibody and a HER2-Fc protein for detection of CD19.scFv and HER2.scFv, respectively (the labeling strategy is outlined in FIG. 8; results are shown in FIG. 3B). After retroviral transduction of CD3/CD28-activated T cells using the transient retroviral supernatant obtained from the TanCAR-expressing 293T cells the TanCAR molecule was detectable on the surface of >70% of the lymphocytes as indicated by flow cytometric analysis (FIG. 3C). Specific detection of the FRP5 HER2.scFv fragment (juxtamembrane binding domain) confirmed that the TanCAR extracellular domain was expressed in its entirety on the surface of the packaging cells as well as the T cells.

### Example 6

### TanCAR-Expressing T Cells Distinctly Recognize Each Target Antigen

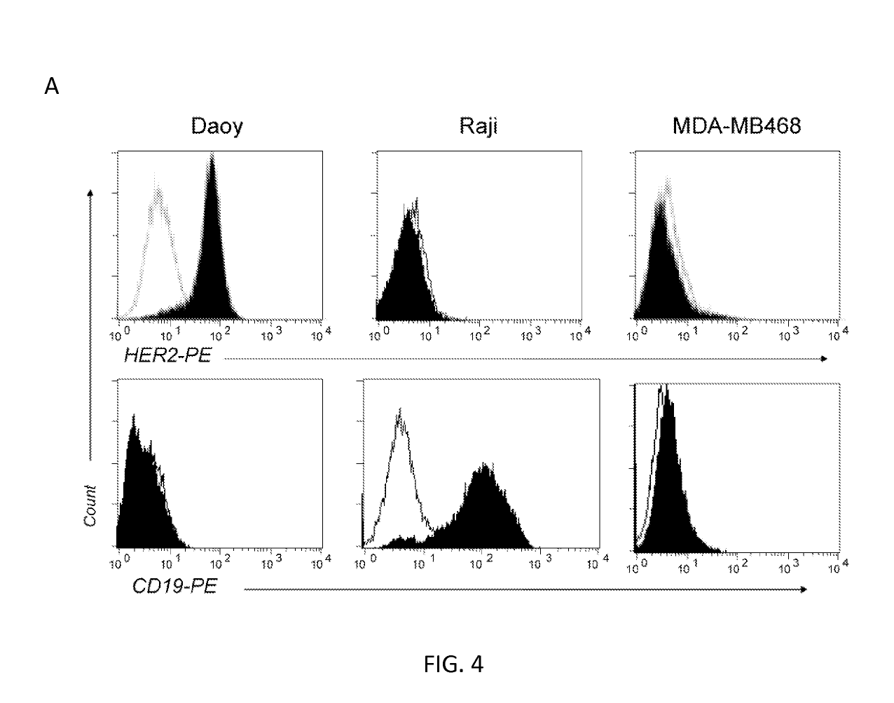

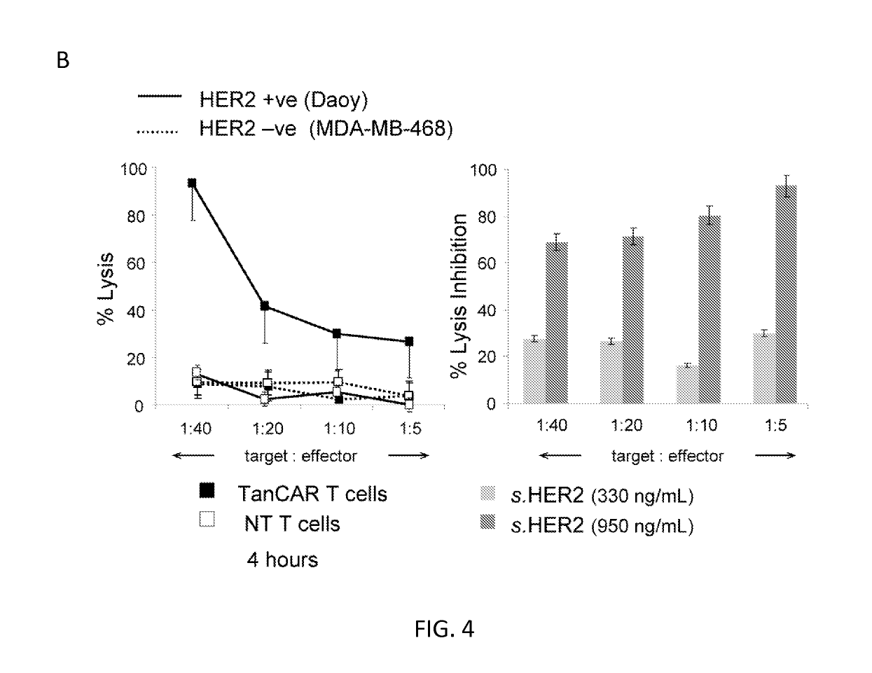

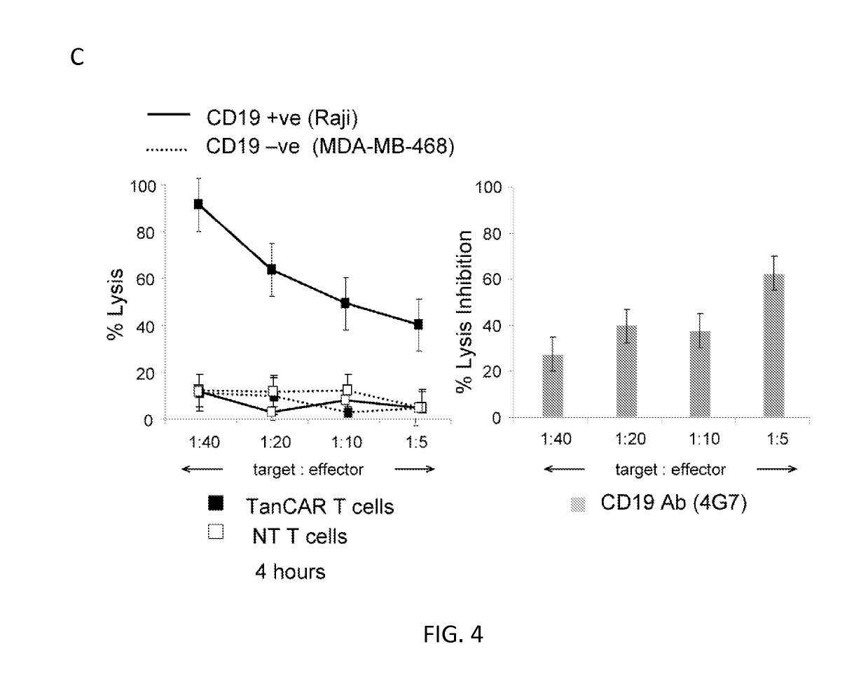

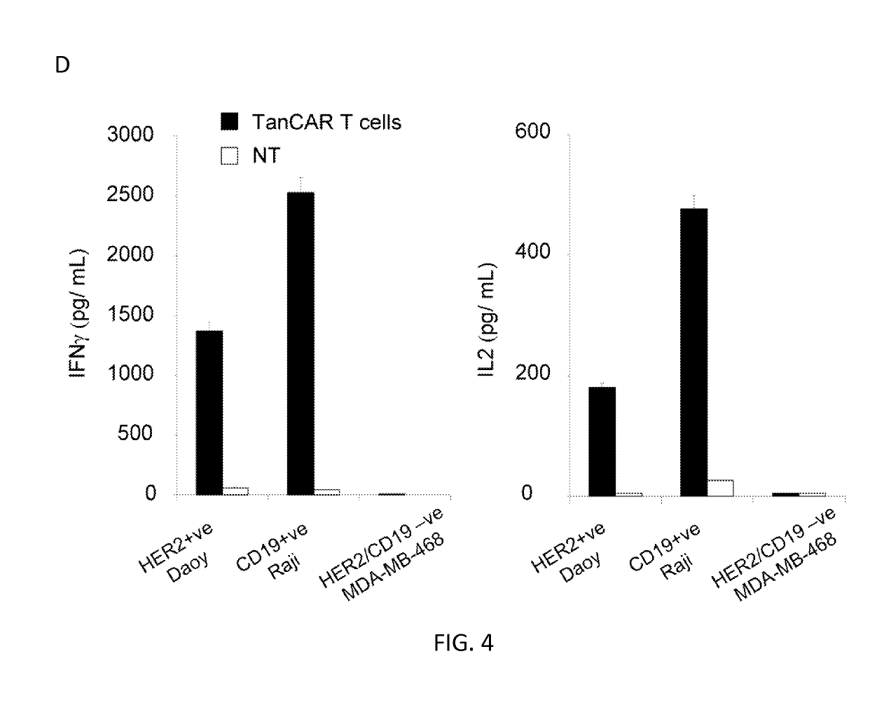

To test the functionality of the bispecific TanCAR against CD19 and HER2, the inventors first confirmed surface expression of these antigens on a panel of human cancer cell lines using flow cytometry. Raji Burkitt lymphoma cells uniformly expressed the B-lineage marker CD19 but lacked detectable HER2 (FIG. 4A) (Savoldo et al., 2011). Conversely, Daoy medulloblastoma cells uniformly expressed HER2 but did not express CD19 (Ahmed et al., 2007). MDA-MB-468 breast cancer cells were negative for both target antigens (Ahmed et al., 2007).

The inventors used this panel of cells to test the dual functionality of the TanCAR. In a 4 hour ⁵¹Cr release assay, TanCAR T cells recognized and killed HER2-expressing Daoy cells but not MDA-MB-468 cells. Non-transduced (NT) T cells from the same donor had no lytic activity, excluding an allogeneic response. Up to 95% of lysis could be blocked by soluble HER2 protein (FIG. 4B) indicating that specific recognition occurred due to HER2 binding. Similarly, CD19-expressing Raji cells were killed by TanCAR T cells but not CD19 negative MDA-MB-468 cells. CD19-specific MAb 4G7 blocked cytolytic activity against Raji cells by up to 65% (FIG. 4C).

In co-cultures, TanCAR T cells secreted increased Interferon-γ (IFNγ) as well as Interleukin-2 (IL-2) after encountering HER2 positive or CD19 positive target cells. No cytokines were secreted in co-culture with HER2 and CD19 negative MDA-MB-468 target cells (FIG. 4D). These results indicate that TanCAR is bispecific for CD19 and HER2, and mediates activation and targeting of T cells upon encounter of either antigen alone.

### Example 7

### Synergistic Effect of Simultaneous Recognition of Both Target Antigens and Preserved TanCAR T Cell-Induced Cytolysis in a Model of Antigen Loss

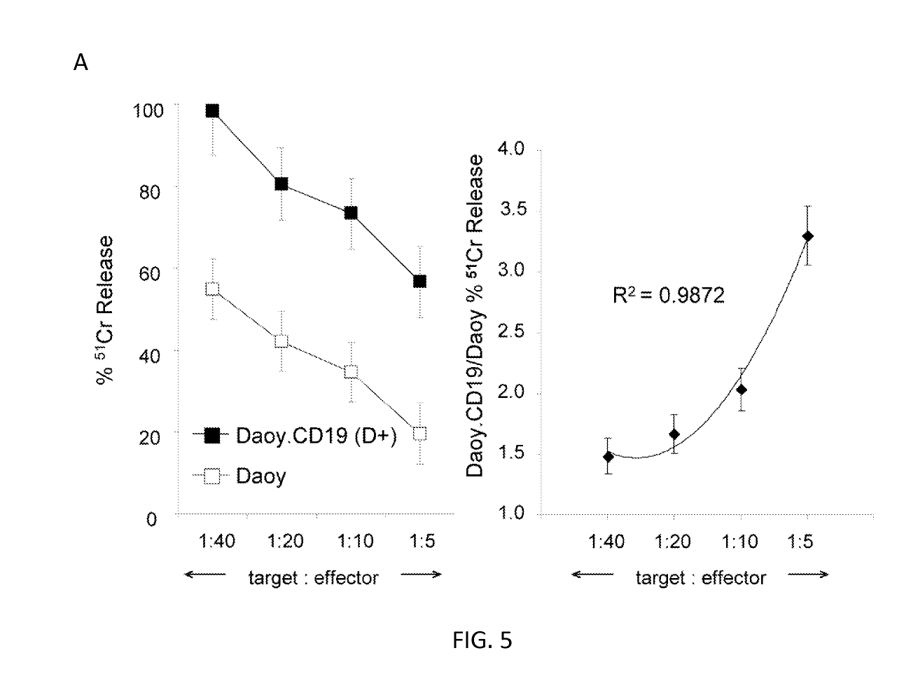

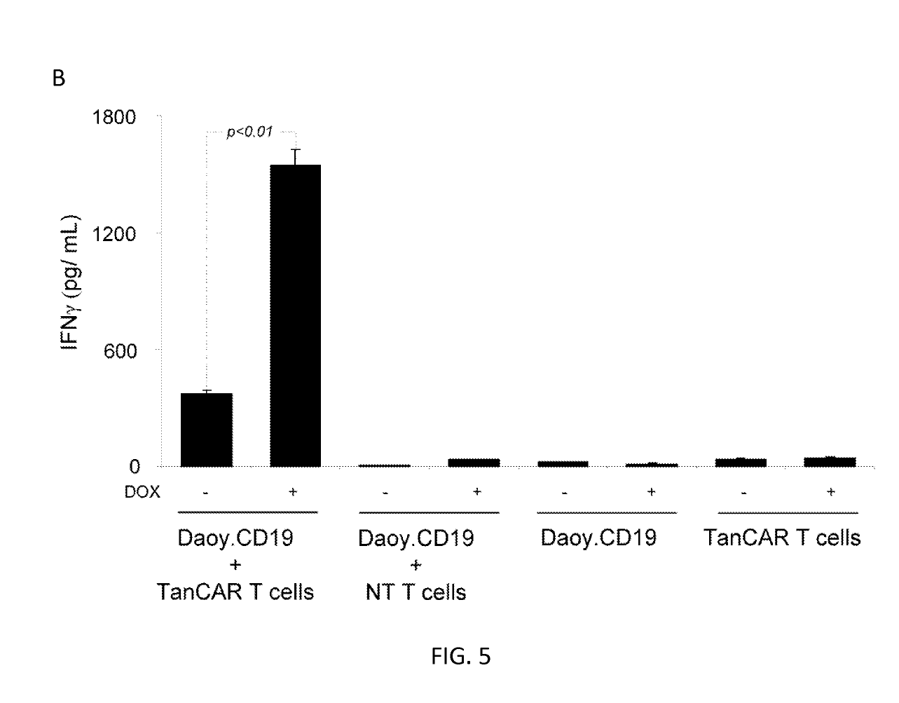

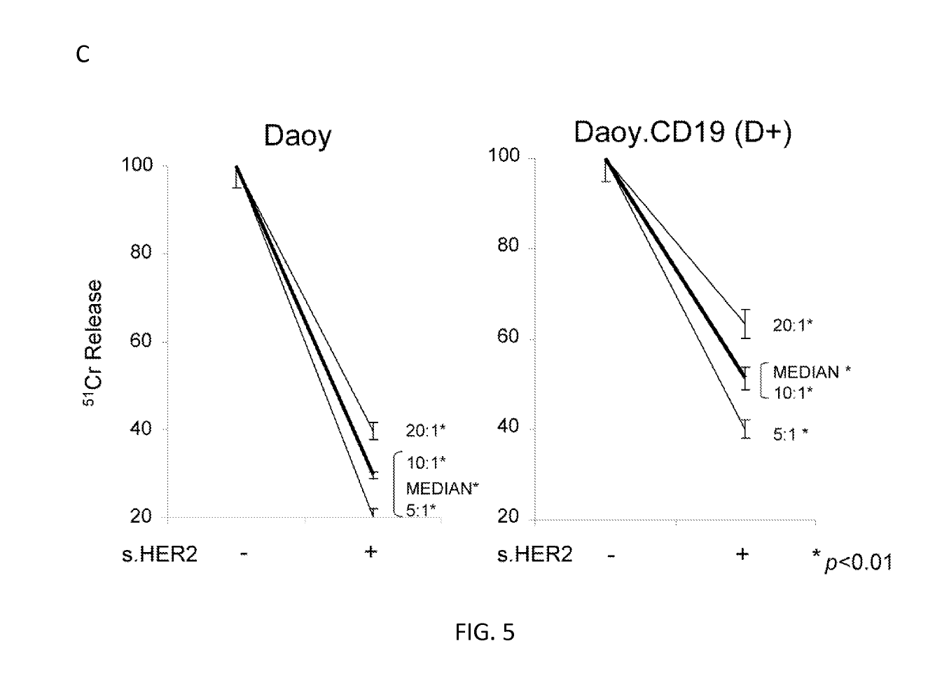

To study the kinetics of activation of TanCAR T cells in the presence of both target molecules, the inventors used a tetracycline inducible system to conditionally express a truncated non-signaling CD19 molecule on Daoy cells (Daoy.TET.CD19) (Zhou et al., 2006). In the presence of Doxycycline (D+), 60-85% of the endogenously HER2 positive Daoy.TET.CD19 cells expressed CD19 and the reporter gene mCherry (FIG. 9). In cytotoxicity assays, there was consistently higher killing after the induction of CD19 at all tumor to T cell ratios tested. The relative difference in lysis in the presence or absence of CD19 followed an exponential trend with a higher order equation, an effect that became more evident at higher tumor to T cell ratios (FIG. 5A), indicating a synergistic contribution of bispecific target cell recognition to effector function. Consistent with these results, there was a four-fold increase (p<0.01) in IFNγ levels in coculture supernatants of TanCAR T cells and Daoy.TET.CD19 cells upon induction of CD19 by the addition of doxycycline (D+) (FIG. 5B).

Downregulation or mutation of target antigens is a common process in cancer cells generating antigen loss escape variants. Such variants are responsible for persistence of tumor cells at very low frequencies, and their outgrowth culminates in relapse. The inventors simulated tumor cell downregulation of a targeted antigen by blocking HER2 in CD19-induced (D+) Daoy.TET.CD19 cells using a soluble HER2 fragment. While the competitor successfully impaired HER2-mediated killing in these cells (up to 90%), bi-specific TanCAR T cells were less affected by soluble HER2 fragment blocking because of maintained recognition of CD19 (FIG. 5C).

### Example 8

### Simultaneous Targeting of Two Antigens Enhances the In Vivo Antitumor Activity of Adoptively Transferred TanCAR T Cells

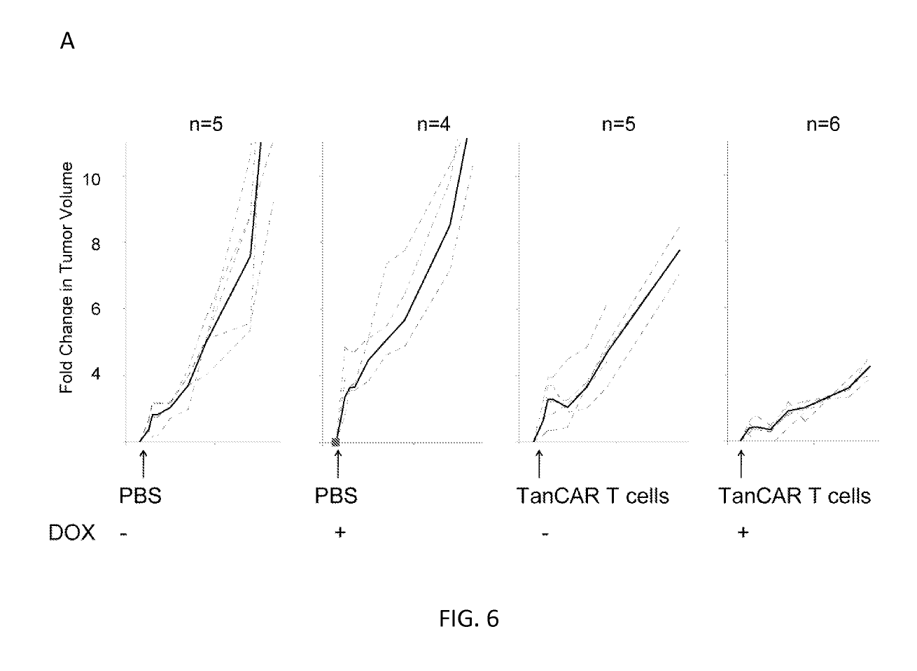

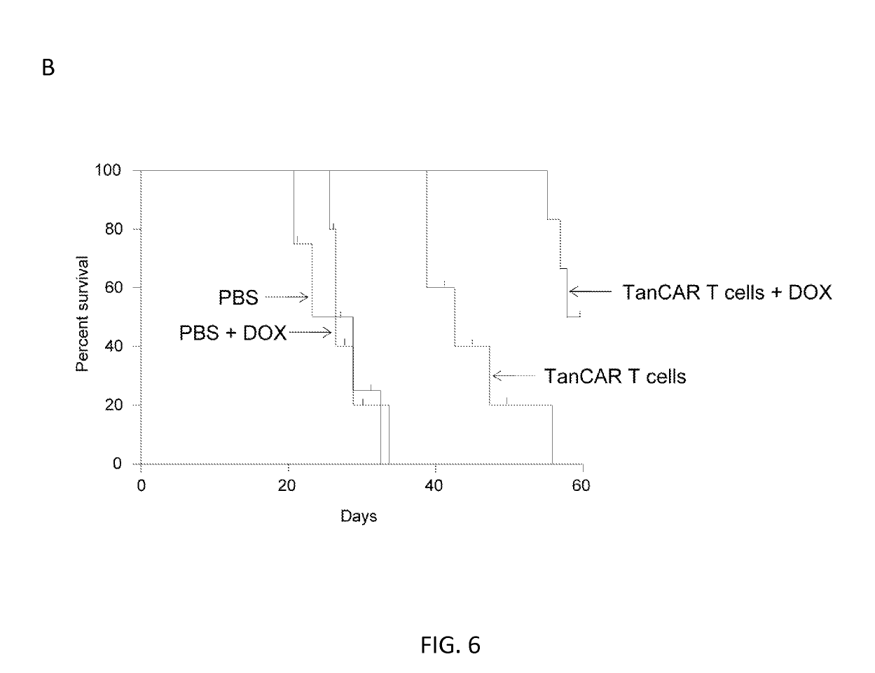

The inventors used a xenograft model to test whether there is an advantage for simultaneous targeting of two antigens in established tumors. Daoy.TET.CD19 xenografts were inoculated in the flanks of SCID mice. The tumors were allowed to establish for approximately 3 weeks, after which animals with established tumors (mean tumor volume 94 μL; SD 28 μL) were randomly assigned into four groups. All four groups of animals had similar tumor volumes after randomization. Two groups received doxycycline by intraperitoneal injection to induce the expression of CD19 (DOX+) while the other two received an equal volume of phosphate-buffered saline (PBS; DOX−). Within D+ and DOX− groups, 5 animals each received intratumoral injections of 10,000 TanCAR T cells/μL, tumor volume. Administration of PBS or doxycycline alone induced minimal or no alteration of the tumor growth pattern. By contrast, treatment with TanCAR T cells resulted in a significant delay in tumor progression that was further increased by induction of CD19 expression in the DOX+ group (FIG. 6A). Kaplan-Meier survival studies 60 days after the PBS or T cells injection showed PBS injected mice with or without doxycycline had a median survival of 28 and 31 days respectively. In contrast, mice treated with TanCAR T cells had a median survival of 44 days (p<0.01). Further, mice treated with TanCAR T cells and receiving doxycycline had a median survival of >60 days with 50% of the mice surviving >80 days (p<0.001; FIG. 6B). Hence, simultaneous recognition of two antigens enhanced the in vivo antitumor activity of adoptively transferred T cells.

### Example 9

### Significance of Embodiments of the Invention

The inventors used computational tools to design and construct a tandem chimeric antigen receptor—TanCAR, a novel artificial molecule that mediates bispecific activation and targeting of T cells. They demonstrated the feasibility of this generally applicable strategy for a complex bispecific TanCAR molecule by cumulative integration of structure and docking simulation data. The prototype TanCAR induced T cell reactivity against each of two target molecules, and produced synergistic enhancement of effector functions when both antigens were simultaneously encountered. Furthermore, the TanCAR preserved the cytolytic effector functions of T cells upon loss of one of the target molecules, and better controlled established experimental tumors by recognition of both targets in an animal disease model.

An approach using effector cells with multiple specificities could have a number of benefits as cancer therapy. First, the simultaneous targeting of multiple tumor antigens could overcome antigen escape by providing an alternative killing pathway if there is antigen down-regulation or deletion. Secondly, such effector cells would exhibit a broader spectrum of specificities allowing for targeting of heterogeneous antigens, such as those present on the tumor cells and within the tumor microenvironment, thereby enhancing tumor control by damaging the tumor complex. Lastly, the encounter of several antigens simultaneously should enhance T cell activation through an increased avidity, a particular benefit when tumors express only modest levels of each target antigen alone (Weijtens et al., 2000; Ahmed et al., 2009). The potential benefits of bispecific antibodies and related molecules have led to an intensive investigation of different designs and specificities of such reagents (Hagemeyer et al., 2009). Unfortunately, their therapeutic use has been hampered by manufacturing feasibility and poor pharmacokinetics and stability (Hagemeyer et al., 2009). Tandem scFv, diabodies, tandem diabodies, two-in-one antibodies, and dual variable domain antibodies (DVD-Ig) are all designs that overcome some of the above limitations but still suffer from the general shortcomings of antibody-based approaches (Hagemeyer et al., 2009; Wu et al., 2007; Gu and Ghayur, 2012; Doppalapudi et al., 2010; Oh et al., 2011). Antibodies—unlike T cells—do not actively migrate through microvascular walls or penetrate the core of solid tumors to exert their antitumor activity and usually have no access to the neuraxis, a common sanctuary for cancer cells. Furthermore, in the context of modest expression of target antigens, antibodies are inefficacious. T cells expressing antibody-derived CARs have been shown to overcome all of these limitations (Weijtens et al., 2000; Ahmed et al., 2010; Ahmed et al., 2009; Verneris et al., 2005).

The group previously redirected the specificity of T cells to two distinct entities by grafting a tumor-restricted antigen-specific CAR onto T cells whose native receptor was specific for latent-virus antigens. Thus Epstein Barr Virus-specific cytotoxic T cells (EBV-CTLs) were grafted with a CAR specific for the disialo-ganglioside GD2 to treat Neuroblastoma, while Cytomegalovirus (CMV)-specific CTLs were grafted with a HER2-specific CAR to treat Glioblastoma (Pule et al., 2008; Study of Administration of CMV-specific Cytotoxic T Lymphocytes Expressing CAR Targeting HER2 I Patients with GBM, 2011). The intent was to enable CAR expressing CTLs to receive optimal costimulation after native-receptor engagement of viral antigens on professional antigen presenting cells, and thereby enhance their in vivo survival in these first-in-man studies (Pule et al., 2008; Study of Administration of CMV-specific Cytotoxic T Lymphocytes Expressing CAR Targeting HER2 I Patients with GBM, 2011). The inventors have now provided bispecificity with a single receptor which should further enhance function and resistance to antigen loss. A combination of these approaches is feasible by grafting a TanCAR to a latent virus specific CTL, thereby providing a trispecific T cell. Such a cell would exhibit dual antitumor activity through its CAR component, and also receive appropriate co-stimulation following native T-cell receptor (αβTCR) engagement by viral antigens presented by APCs. The TanCAR could be further modified to incorporate additional co-stimulatory endodomains to enhance the degree of T cell activation and persistence that follows antigen engagement (Savoldo et al., 2011; Porter et al., 2011).

Though much progress has been made, designing novel protein molecules with correct protein folds is still very challenging (Buchner et al., 2011; Kuhlman and Baker, 2004). However, advances in computational modeling and protein-protein docking have made structure design and analysis of a novel protein, such as the aforementioned tandem CAR, more feasible (Park et al., 2004; Perez-Aguilar and Saven, 2012; Samish et al., 2011). By combining biochemical data and employing computational tools, the inventors were able to generate a structural model for TanCAR, as well as model its interface with two target antigens. This TanCAR was then expressed in T cells and analyzed for its biological activity.

The invention describes the first artificial molecule to render T cells bi-specific. The inventors used modern computational modeling and docking tools, in a staged methodology, to profile the energy landscapes as well as the dynamics of the polypeptide chains in all stages of the folding process, making the design of a bi-specific CAR more feasible. The TanCAR model was produced from several closely related structures (>50% sequence identity between the sequences of interest and the template structures), demonstrating that computational modeling is a useful tool in the construction of such complex molecules. However, the Gly-Ser linker has no structural template; therefore, the exact structure of this loop and the orientations of the two TanCAR domains with respect to each other most likely exhibit some degree of variation. It is conceivable that, while unbound, TanCAR remains as a soluble, compact, globular protein. However, when bound to HER2 and CD19, steric forces may cause the two domains to separate, remaining tethered to each other through an extended conformation of the Gly-Ser linker. In an extended conformation, this loop could help resolve some of the differences in “height” of the two target molecules on the surface of the cell. Regardless of the conformation of the loop, the interface proposed by the docking is compatible with either a compact or extended form of the linker.

Indeed, when the hypothetical molecule was physically generated and tested, the results corroborated the predictive modeling. The surface expression of the optimized TanCAR exodomain in its entirety was validated by flow cytometry with a detection strategy that was specific to the juxta-membrane portion of the molecule (using HER2—Fc as a HER2.scFv (FRP5)-specific moiety). The proper folding and retention of the V_{L} and V_{H} stereo-orientation for HER2 scFv (FRP5) and CD19 scFv was evident in the specific recognition and distinct lysis by TanCAR T cells of tumor cells expressing these target molecules and the blockade of this lysis with the respective competitors. Failure to block the cytolytic effect against cells expressing both targets using soluble HER2 indicated that the dual functionality of TanCAR T cells allowed for persistence of their effector functions despite antigen loss. This redundancy in function would be favorable in such a scenario. Lastly, the exponential enhancement of cytolysis upon induction of CD19 in HER2 expressing target cells indicated synergistic dynamics that further translated into improved tumor control in an animal model of an established tumor using a relatively small dose of T cells. These findings indicate the utility of TanCAR T cells for therapeutic application in human disease.

With the enhanced activation of TanCAR T cells comes the risk of adverse events related to recognition of modestly expressed antigens resulting in off-target effects. While it has been recently shown that it is possible to quickly eliminate adoptively transferred T cells in case of adverse events by inducing apoptosis through a suicide gene (Di et al., 2011)—TanCAR molecules may be used to “bar code” target cells—wherein only dual antigen expressers are recognized and killed. Alternatively, TanCAR molecules themselves are engineered to include antibody recognizable moieties that—in the advent of adverse events—would allow for the rapid elimination of TanCAR T cells, in specific embodiments of the invention.

In summary, the inventors provide an approach for a novel bispecific chimeric antigen receptor molecule—termed TanCAR. The TanCAR induced bispecific activation of T cells and exhibited potent effector functions against individual target antigens as well as synergistic enhancement of functionality upon simultaneous engagement of both. Preclinical studies of TanCAR T cells in an animal tumor model demonstrated its utility for therapeutic application in human disease.

### Example 10

### Materials and Methods

Blood Donors, Primary Tumor Cells and Cell Lines.

Studies were performed on Baylor College of Medicine IRB-approved protocols and informed consent was obtained from all donors. The medulloblastoma line Daoy was purchased from ATCC (Manassas, Va.). All cell lines were grown in DMEM (Invitrogen, Carlsbad, Calif.) with 10% fetal calf serum (FCS; HyClone, Logan, Utah), with 2 mM GlutaMAX-I, 1.5 g/L sodium bicarbonate and 1.0 mmol/L sodium pyruvate (Invitrogen). T cells derived from PBMCs were activated on CD3CD28 antibody-coated plates and were expanded in IL-2 (100 U/mL)-containing RPMI 1640 with 10% FCS and 2 mM GlutaMAX-I. Tumor cells were grown in supplemented DMEM.

Protein Structure Modeling and Docking.

A model for the bi-specific CAR was constructed using ModWeb, an automated web server for protein structure modeling (Pieper et al., 2011; Eswar et al., 2003). The full length TanCAR sequence was submitted to the ModWeb server, which identified the two antibody-like fragment domains. 10P3 (Calarese et al., 2003) was identified as a candidate structural template for residues 40-146 (59.81% sequence identity) and 1F3R (Kleinjung et al., 2000) as a candidate structural template for residues 167-329 (58.90% sequence identity). Additional structural templates, 3ESV (64.76% sequence identity) and 2 KH2 (59.51% sequence identity) (Leysath et al., 2009; Wilkinso et al., 2009), were also identified covering residues 38-285. From these templates, a homology model was constructed spanning residues 39-329, including the 20 amino acid long Gly-Ser linker. The model was truncated at residue 285 as residues 286-329 were poorly modeled. The structure for HER2 was available (PDB ID: 1N8Z) (Cho et al., 2003). As a note, the 1N8Z structure contains residues 23-629, resulting in a difference in numbering between it and the HER2 precursor protein sequence (uniprot P04626) (Yamamoto et al., 1986). A homologous structure for CD19 was identified using the BioInfoBank metaserver (Ginalski et al., 2003). A model for residues 1-272 CD19 was constructed with Modeller V9.1 (Eswar et al., 2003) using 3MJG (12.59% sequence identity) as a structural template (Shim et al., 2010).

Initially, pairwise docking was performed with PatchDock (Schnediman-Duhovny et al., 2005) using the individual TanCAR domains and the corresponding receptor; residues 39-155 corresponded to the HER2 binding portion of TanCAR, while residues 156-285 were assigned to the CD19 binding portion of TanCAR. Fits were evaluated visually and based on their PatchDock score. For the TanCAR-HER2 docking, results were additionally filtered based on peptide spotting experiments that had suggested binding residues (FIG. 7). Further refinement of the individual candidate dockings was done using FireDock (Andrusier et al., 2007). Candidate dockings from both CAR-HER2 and CAR-CD19 were then combined in UCSF Chimera (Pettersen et al., 2004) and evaluated for steric clashes. The final model for the CD19-CAR-HER2 docking was selected based on lowest global energy in each of the pairwsie dockings from FireDock and steric constraints in the entire assembly.

Construction, Delivery and Expression of the TanCAR Encoding Transgene.

The scFv domain targeting the CD19 antigen was provided by Heddy Zola (Child Health Research Institute, Women's and Children's Hospital, Adelaide, South Australia, Australia) (Zola et al., 1991). The scFv domain targeting HER2 (FRP5) was previously described by Wels and colleagues (Wels et al., 1992). The modeled bi-specific extracellular domain (excluding the Gly-Ser tandem repeat hinge), composed of the CD19 and HER2 scFv antibody fragments in tandem and separated by a linker, was assembled on Clone Manager® (Sci-Ed Software Inc, Cary, N.C.). The designed transgene DNA sequence was modified to include restriction enzyme sites at the cloning sites and exclude any inadvertently inserted sites within the translation elements, then optimized using the GeneOptimizer® software for maximum protein production (Raab et al., 2010). The TanCAR extracellular domain was then synthesized by by GeneArt® Inc. using oligonucleotides, cloned into the Gateway® entry vector pDONR™221, standard cloning vector, and sequence-verified. This antigen recognition domain was then subcloned in frame into an SFG retroviral vector containing a short hinge, and the transmembrane and signaling domain of the costimulatory molecule, CD28 and the signaling domain of the T-cell receptor—chain (FIG. 3A) (Moritz et al., 1994; Rossig et al., 2001; Pule et al., 2005). The structure of the construct was confirmed using restriction digests. The 5′-and 3′ as well as the 3′-5′ sequence of the whole construct was confirmed using single base pair pyro-sequencing (SeqWright DNA Technology Services, Houston, Tex.) with a homology of >97% with the optimized construct map.

Retrovirus Production and Transduction of T Cells

To produce retroviral supernatant, human embryonic kidney (HEK) 293T cells were co-transfected with the TanCAR-encoding retroviral transfer plasmid, Peg-Pam-e plasmid encoding MoMLV gag-pol, and plasmid pMEVSVg containing the sequence for VSV-G envelope (Ahmed et al., 2007), using GeneJuice transfection reagent (EMD Biosciences, San Diego, Calif.) (Rainusso et al., 2011). Supernatants containing retroviral vector were collected 48 and 72 hours later.

Anti-CD3 (OKT3)/anti-CD28 activated T cells were transduced with retroviral vectors as described (Vera et al., 2006). Briefly, peripheral blood mononuclear cells (PBMC) were isolated by Lymphoprep (Greiner Bio-One, Monroe, N.C.) gradient centrifugation. 5×10⁵ PBMC per well in a 24-well plate were activated with OKT3 (OrthoBiotech, Raritan, N.J.) and CD28 monoclonal antibodies (BD Biosciences, Palo Alto, Calif.) at a final concentration of 1 μg/mL. On day 2, recombinant human IL-2 (Chiron, Emeryville, Calif.) was added at a final concentration of 100 U/mL, and two days later, cells were harvested for retroviral transduction. For transduction, we pre-coated a non-tissue culture treated 24-well plate with a recombinant fibronectin fragment (FN CH-296; Retronectin; Takara Bio USA, Madison, Wis.). Wells were washed with phosphate-buffered saline (PBS; Sigma, St. Louis, Mo.) and incubated twice for 30 minutes with vector particles. Subsequently, 3×10⁵ T cells per well were transduced with retrovirus in the presence of 100 U/mL IL-2. After 48-72 hours cells were removed and expanded in the presence of 50-100 U/mL IL-2 for 10-15 days prior to use.

Cytotoxicity Assays

Cytotoxicity assays were performed as previously described (Gottschalk et al., 2003). Briefly, 1×10⁶ target cells were labeled with 0.1 mCi (3.7 MBq) ⁵¹Cr and mixed with decreasing numbers of effector cells to give effector to target ratios of 40:1, 20:1, 10:1 and 5:1. Target cells incubated in complete medium alone or in 1% Triton X-100 were used to determine spontaneous and maximum ⁵¹Cr release, respectively. After 4 hours we collected supernatants and measured radioactivity in a gamma counter (Cobra Quantum; PerkinElmer; Wellesley; MA). The mean percentage of specific lysis of triplicate wells was calculated according to the following formula: [test release−spontaneous release]/[maximal release−spontaneous release]×100.

Analysis of Cytokine Production and T-Cell Expansion

Effector T cells (TanCAR expressing T cells or non-transduced T cells) from healthy volunteers were co-cultured with tumor cells in short-term culture, HER2-positive and HER2-negative cell lines, at various effector to target ratios in a 24 well plate. After 24 to 48 hours incubation, culture supernatants were harvested and the presence of IFN-γ and IL-2 was determined by ELISA as per the manufacturer's instructions (R&D Systems, Minneapolis, Minn.). T-cell expansion was determined by counting viable cells (trypan blue exclusion) seven days after stimulation.

Tetracycline-Inducible System.

To express the truncated CD19 (CD19) protein on Daoy cells, we used the Tet-On® 3G Tetracycline-Inducible Expression System (CloneTech, Mountainview, Calif.). The CD 19 encoding DNA fragment was subcloned downstream of the inducible promoter P_{TRE3G} using PCR amplification. Daoy cells expressed CD 19, but only when cultured in the presence of doxycycline (Dox), a tetracycline analog. When bound by Dox, the Tet-On® 3G protein undergoes a conformational change that allows it to bind to tet operator (tetO) sequences located in the inducible promoter P_{TRE3G}. The addition of doxycycline also initiated a proportionate expression of the reporter gene mCherry.

Flow Cytometry.

For all flow cytometric analyses, a FACScalibur instrument (BD, Becton Dickinson, Mountain View, Calif.) and CellQuest software (BD) were used. Data analysis was done on >10,000 events; in all cases negative controls included isotype antibodies. Cells were washed once with PBS containing 2% FBS and 0.1% sodium azide (Sigma; FACS buffer) prior to addition of antibodies. After 15 to 30 minutes of incubation at 4° C. in the dark the cells were washed once and fixed in 0.5% paraformaldehyde/FACS buffer prior to analysis. T cells were analyzed with anti-CD8 FITC, -CD4 PE, and -CD3 PerCP. All monoclonal antibodies were obtained from BD Biosciences, Palo Alto, Calif. Surface expression of the TanCAR was assessed using a HER2 scFv (FRP5)-specific method by incubation with a soluble HER2.Fc fragment followed by a human Fc specific FITC-labeled antibody. Alternatively, APC-conjugated Fab-specific antibody was used to detect either HER2 scFv (FRP5) or CD19 scFv.

Animal Studies.

All animal experiments were conducted on a protocol approved by the Baylor College of Medicine Institutional Animal Care and Use Committee. Recipient non-obese diabetic-severe combined immunodeficiency (NOD-SCID) mice were purchased from Taconic (C.B-Igh-1b/IcrTac-Prkdcscid; FOX CHASE CB-17 SCID™ ICR; Taconic, Hudson, N.Y.). Eight to ten week old male mice were anesthetized with rapid sequence inhalation of isofluorane (Abbot Laboratories) followed by subcutaneous injection of 1×10⁶ DAOY.TET.CD19 cells (in 100 μl PBS) per flank per mouse (Day 0). Tumors were then allowed approximately three weeks to fully engraft. Mice with established tumors (mean tumor volume 940: SD 28 μL) were randomly assigned to four groups (n=5 tumors/group). Tumor volume was calculated from the product of the bi-dimensional area measured with an electronic caliper. All four groups of animals had similar tumor volumes after randomization. To induce the expression of CD 19 (D+) two groups received daily for one week, 2400 μg/kg of doxycycline via intraperitoneal injection (individual dose of 80 μg/mouse) followed by one daily dose three times a week. The other half received an equal volume of PBS (D-) on the same dosing schedule. On the third day of doxycycline/PBS administration, within the D+ and D-groups, 5 animals each received intratumoral injections of 10,000 TanCAR T cells per μL tumor volume. All mice were then blindly assessed for changes in tumor volume.

Statistical Analysis.

The Student's t test was used to test for significance in each set of values, assuming equal variance. Mean values plus or minus SDs are given unless otherwise stated.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### PUBLICATIONS

Ahmed, N. et al. Immunotherapy for osteosarcoma: genetic modification of T cells overcomes low levels of tumor antigen expression. Mol. Ther. 17, 1779-1787 (2009).
Ahmed, N. et al. Regression of experimental medulloblastoma following transfer of HER2-specific T cells. Cancer Res. 67, 5957-5964 (2007).
Ahmed, N., Salsman V S, Kew Y et al. HER2-specific T cells target primary glioblastoma stem cells and induce regression of autologous experimental tumors. Clin. Cancer Res. 2010; 16:474-485.
Andrusier, N., Nussinov, R., & Wolfson, H. J. FireDock: fast interaction refinement in molecular docking. Proteins 69, 139-159 (2007).
Baillie, R., Harada K, Carlile J et al. Expression of vascular endothelial growth factor in normal and tumour oral tissues assessed with different antibodies. Histochem. J. 2001; 33:287-294.
Barbera-Guillem, E., Nyhus J K, Wolford C C, Friece C R, Sampsel J W. Vascular endothelial growth factor secretion by tumor-infiltrating macrophages essentially supports tumor angiogenesis, and IgG immune complexes potentiate the process. Cancer Res. 2002; 62:7042-7049.
Bergers, G., Coussens L M. Extrinsic regulators of epithelial tumor progression: metalloproteinases. Curr. Opin. Genet. Dev. 2000; 10:120-127.
Bergers, G., Hanahan D, Coussens L M. Angiogenesis and apoptosis are cellular parameters of neoplastic progression in transgenic mouse models of tumorigenesis. Int. J. Dev. Biol. 1998; 42:995-1002.
Bissell, M. J., Kenny P A, Radisky D C. Microenvironmental regulators of tissue structure and function also regulate tumor induction and progression: the role of extracellular matrix and its degrading enzymes. Cold Spring Harb. Symp. Quant. Biol. 2005; 70:343-356.
Bollard, C. M., Aguilar L, Straathof K C et al. Cytotoxic T Lymphocyte Therapy for Epstein-Barr Virus+Hodgkin's Disease. J. Exp. Med. 2004; 200:1623-1633.
Bollard, C. M., Gottschalk S, Leen A M et al. Complete responses of relapsed lymphoma following genetic modification of tumor-antigen presenting cells and T-lymphocyte transfer. Blood 2007; 110:2838-2845.
Brentjens, R. J., et al. Eradication of systemic B-cell tumors by genetically targeted human T lymphocytes co-stimulated by CD80 and interleukin-15. Nat. Med. 9, 279-286 (2003).
Buchner, G. S., Murphy, R. D., Buchete, N. V., & Kubelka, J. Dynamics of protein folding: probing the kinetic network of folding-unfolding transitions with experiment and theory. Biochim. Biophys. Acta 1814, 1001-1020 (2011).
Calarese, D. A. et al. Antibody domain exchange is an immunological solution to carbohydrate cluster recognition. Science 300, 2065-2071 (2003).
Cho, H. S., et al. Structure of the extracellular region of HER2 alone and in complex with the Herceptin Fab. Nature 421, 756-760 (2003).
Currie, M. J., Hanrahan V, Gunningham S P et al. Expression of vascular endothelial growth factor D is associated with hypoxia inducible factor (HIF-1alpha) and the HIF-1alpha target gene DEC1, but not lymph node metastasis in primary human breast carcinomas. J. Clin. Pathol. 2004; 57:829-834.
Di, S. A. et al. Inducible apoptosis as a safety switch for adoptive cell therapy. N. Engl. J. Med. 365, 1673-1683 (2011).
Doppalapudi, V. R. et al. Chemical generation of bispecific antibodies. Proc. Natl. Acad. Sci. U.S. A 107, 22611-22616 (2010).
Dotti, G., Savoldo B, Pule M et al. Human cytotoxic T lymphocytes with reduced sensitivity to Fasinduced apoptosis. Blood 2005; 105:4677-4684.
Dotti, G., Savoldo, B., & Brenner, M. Fifteen years of gene therapy based on chimeric antigen receptors: “are we nearly there yet?”. Hum. Gene Ther. 20, 1229-1239 (2009).
Dudley, M. E., Wunderlich J R, Robbins P F et al. Cancer regression and autoimmunity in patients after clonal repopulation with antitumor lymphocytes. Science 2002; 298:850-854.
Dudley, M. E., Wunderlich J R, Yang J C et al. Adoptive cell transfer therapy following non-myeloablative but lymphodepleting chemotherapy for the treatment of patients with refractory metastatic melanoma. J. Clin Oncol. 2005; 23:2346-2357.
Dudley, M. E., Yang J C, Sherry R et al. Adoptive cell therapy for patients with metastatic melanoma: evaluation of intensive myeloablative chemoradiation preparative regimens. J. Clin. Oncol. 2008; 26:5233-5239.
Dunn, G. P., Old, L. J., & Schreiber, R. D. The three Es of cancer immunoediting. Annu. Rev. Immunol. 22, 329-360 (2004).
Egeblad, M., Nakasone E S, Werb Z. Tumors as organs: complex tissues that interface with the entire organism. Dev.Cell 2010; 18:884-901.
Eshhar, Z., Waks, T., Gross, G., & Schindler, D. G. Specific activation and targeting of cytotoxic lymphocytes through chimeric single chains consisting of antibody-binding domains and the gamma or zeta subunits of the immunoglobulin and T-cell receptors. Proc. Natl. Acad. Sci. U.S. A 90, 720-724 (1993).
Esposito, I., Menicagli M, Funel N et al. Inflammatory cells contribute to the generation of an angiogenic phenotype in pancreatic ductal adenocarcinoma. J. Clin. Pathol. 2004; 57:630-636.
Eswar, N., et al. Tools for comparative protein structure modeling and analysis. Nucleic Acids Res. 31, 3375-3380 (2003).
Gerstmayer, B., Altenschmidt, U., Hoffmann, M., & Wels, W. Costimulation of T cell proliferation by a chimeric B7-2 antibody fusion protein specifically targeted to cells expressing the erbB2 proto-oncogene. J. Immunol. 158, 4584-4590 (1997).
Ginalski, K., Elofsson, A., Fischer, D., & Rychlewski, L. 3D-Jury: a simple approach to improve protein structure predictions. Bioinformatics. 19, 1015-1018 (2003).
Gottschalk, S, Ng C Y C, Smith C A et al. An Epstein-Barr virus deletion mutant that causes fatal lymphoproliferative disease unresponsive to virus-specific T cell therapy. Blood 2001; 97:835-843.
Gottschalk, S. et al. Generating CTL against the subdominant Epstein-Barr virus LMP1 antigen for the adoptive Immunotherapy of EBV-associated malignancies. Blood 101, 1905-1912 (2003).
Gottschalk, S., Heslop H E, Rooney C M. Adoptive immunotherapy for EBV-associated malignancies. Leuk. Lymphoma 2005; 46:1-10.
Gross, G, Gorochov G, Waks T, Eshhar Z. Generation of effector T cells expressing chimeric T cell receptor with antibody type-specificity. Transplant. Proc. 1989; 21:127-130.
Gu, J. & Ghayur, T. Generation of dual-variable-domain immunoglobulin molecules for dual-specific targeting. Methods Enzymol. 502, 25-41 (2012).

Hagemeyer, C. E., von Zur, M. C., von, E. D., & Peter, K. Single-chain antibodies as diagnostic tools and therapeutic agents. Thromb. Haemost. 101, 1012-1019 (2009).
Hanrahan, V., Currie M J, Gunningham S P et al. The angiogenic switch for vascular endothelial growth factor (VEGF)-A, VEGF-B, VEGF-C, and VEGF-D in the adenoma-carcinoma sequence during colorectal cancer progression. J. Pathol. 2003; 200:183-194.
Her2 and TGFBeta in Treatment of Her2 Positive Lung Malignancy. 2011.
Her2 Chimeric Antigen Receptor Expressing T Cells in Advanced Osteosarcoma. 2011.
Heslop, H. E., Ng C YC, Li C et al. Long-term restoration of immunity against Epstein-Barr virus infection by adoptive transfer of gene-modified virus-specific T lymphocytes. Nature Medicine 1996; 2:551-555.
Hunder, N, N., Wallen H, Cao J et al. Treatment of metastatic melanoma with autologous CD4+ T cells against NY-ESO-1. N. Engl. J. Med. 2008; 358:2698-2703.
Kalos, M. et al. T cells with chimeric antigen receptors have potent antitumor effects and can establish memory in patients with advanced leukemia. Sci. Transl. Med. 3, 95ra73 (2011).
Kiel, C., Beltrao, P., & Serrano, L. Analyzing protein interaction networks using structural information. Annu. Rev. Biochem. 77, 415-441 (2008).
Kleinjung, J., et al. The third-dimensional structure of the complex between an Fv antibody fragment and an analogue of the main immunogenic region of the acetylcholine receptor: a combined two-dimensional NMR, homology, and molecular modeling approach. Biopolymers 53, 113-128 (2000).
Kuhlman, B. & Baker, D. Exploring folding free energy landscapes using computational protein design. Curr. Opin. Struct. Biol. 14, 89-95 (2004).
Leysath, C. E., et al. Crystal structure of the engineered neutralizing antibody M18 complexed to domain 4 of the anthrax protective antigen. J. Mol. Biol. 387, 680-693 (2009).
Loskog, A., Dzojic H, Vikman S et al. Adenovirus CD40 ligand gene therapy counteracts immune escape mechanisms in the tumor Microenvironment. J. Immunol. 2004; 172:7200-7205.
Louis, C. U., Straathof K, Bollard C M et al. Enhancing the in vivo expansion of adoptively transferred EBV-specific CTL with lymphodepleting CD45 monoclonal antibodies in NPC patients. Blood 2008
Marcotte, E. M., Pellegrini, M., Yeates, T. O., & Eisenberg, D. A census of protein repeats. J. Mol. Biol. 293, 151-160 (1999).
Matsushima, N., et al. Flexible structures and ligand interactions of tandem repeats consisting of proline, glycine, asparagine, serine, and/or threonine rich oligopeptides in proteins. Curr. Protein Pept. Sci. 9, 591-610 (2008).
Morgan, R. A., Dudley M E, Wunderlich J R et al. Cancer regression in patients after transfer of genetically engineered lymphocytes. Science 2006; 314:126-129.
Moritz, D., Wels, W., Mattern, J., & Groner, B. Cytotoxic T lymphocytes with a grafted recognition specificity for ERBB2-expressing tumor cells. Proc. Natl. Acad. Sci. U.S. A 91, 4318-4322 (1994).
Nakazawa, Y. et al. PiggyBac-mediated cancer immunotherapy using EBV-specific cytotoxic T-cells expressing HER2-specific chimeric antigen receptor. Mol. Ther. 19, 2133-2143 (2011).
Oh, S., Tsai, A. K., Ohlfest, J. R., Panoskaltsis-Mortari, A., & Vallera, D. A. Evaluation of a bispecific biological drug designed to simultaneously target glioblastoma and its neovasculature in the brain. J. Neurosurg. 114, 1662-1671 (2011).
Pakakasama, S., Eames G M, Morriss M C et al. Treatment of Epstein-Barr Virus Lymphoproliferative Disease after Hematopoietic Stem-Cell Transplantation with Hydroxyurea and Cytotoxic T-Cell Lymphocytes. Transplantation 2004; 78:755-757.
Park, J. R., Digiusto D L, Slovak M et al. Adoptive transfer of chimeric antigen receptor re-directed cytolytic T lymphocyte clones in patients with neuroblastoma. Mol. Ther. 2007; 15:825-833.
Park, S., Yang, X., & Saven, J. G. Advances in computational protein design. Curr. Opin. Struct. Biol. 14, 487-494 (2004).
Perez-Aguilar, J. M. & Saven, J. G. Computational design of membrane proteins. Structure. 20, 5-14 (2012).
Pettersen, E. F., et al. UCSF Chimera—a visualization system for exploratory research and analysis. J. Comput. Chem. 25, 1605-1612 (2004).
Pieper, U. et al. ModBase, a database of annotated comparative protein structure models, and associated resources. Nucleic Acids Res. 39, D465-D474 (2011).
Porter, D. L., Levine B L, Kalos M, Bagg A, June C H. Chimeric antigen receptor-modified T cells in chronic lymphoid leukemia. N. Engl. J. Med. 2011; 365:725-733.
Pule, M. et al. Three-Module Signaling Endo-Domain Artificial T-cell Receptor which transmits CD28, OX40 and CD3-zeta signals enhances IL-2 release and proliferative response in transduced primary T-cells. Blood 104, 485a (2004).
Pule, M., Finney, H., & Lawson, A. Artificial T-cell receptors. Cytotherapy. 5, 211-226 (2003).
Pule, M. A., Savoldo B, Myers G D et al. Virus-specific T cells engineered to coexpress tumor-specific receptors: persistence and antitumor activity in individuals with neuroblastoma. Nat Med 2008; 14:1264-1270.
Pule, M. A., Straathof K C, Dotti G et al. A chimeric T cell antigen receptor that augments cytokine release and supports clonal expansion of primary human T cells. Mol.Ther. 2005.
Raab, D., Graf, M., Notka, F., Schodl, T., & Wagner, R. The GeneOptimizer Algorithm: using a sliding window approach to cope with the vast sequence space in multiparameter DNA sequence optimization. Syst. Synth. Biol. 4, 215-225 (2010).
Rainusso, N., et al. Immunotherapy targeting HER2 with genetically modified T cells eliminates tumor-initiating cells in osteosarcoma. Cancer Gene Ther.(2011).
Riddell, S. R., Watanabe K S, Goodrich J M et al. Restoration of viral immunity in immunodeficient humans by the adoptive transfer of T cell clones. Science 1992; 257:238-241.
Robinson, M. K. et al. Targeting ErbB2 and ErbB3 with a bispecific single-chain Fv enhances targeting selectivity and induces a therapeutic effect in vitro. Br. J. Cancer 99, 1415-1425 (2008).
Rooney, C. M., Smith C A, Ng C Y C et al. Infusion of cytotoxic T cells for the prevention and treatment of Epstein-Barr virus-induced lymphoma in allogeneic transplant recipients. Blood 1998; 92:1549-1555.
Rosenberg, S. A., Restifo N P, Yang J C, Morgan R A, Dudley M E. Adoptive cell transfer: a clinical path to effective cancer immunotherapy. Nat. Rev. Cancer 2008; 8:299-308.
Rossig, C., Bollard C M, Nuchtern J G, Merchant D A, Brenner M K. Targeting of G(D2)-positive tumor cells by human T lymphocytes engineered to express chimeric T-cell receptor genes. Int J Cancer 2001; 94:228-236.
Samish, I., MacDermaid, C. M., Perez-Aguilar, J. M., & Saven, J. G. Theoretical and computational protein design. Annu. Rev. Phys. Chem. 62, 129-149 (2011).
Savoldo, B., et al. Epstein Barr virus specific cytotoxic T lymphocytes expressing the anti-CD30zeta artificial chimeric T-cell receptor for immunotherapy of Hodgkin disease. Blood 110, 2620-2630 (2007).
Savoldo, B., Ramos C A, Liu E et al. CD28 costimulation improves expansion and persistence of chimeric antigen receptor-modified T cells in lymphoma patients. J. Clin. Invest 2011; 121:1822-1826.
Schneidman-Duhovny, D., Inbar, Y., Nussinov, R., & Wolfson, H. J. PatchDock and SymmDock: servers for rigid and symmetric docking. Nucleic Acids Res. 33, W363-W367 (2005).
Shim, A. H., et al. Structures of a platelet-derived growth factor/propeptide complex and a platelet-derived growth factor/receptor complex. Proc. Natl. Acad. Sci. U.S. A 107, 11307-11312 (2010).
Straathof, K. C., Bollard C M, Popat U et al. Treatment of Nasopharyngeal Carcinoma with Epstein-Barr Virus-specific T Lymphocytes. Blood 2005; 105:1898-1904.
Straathof, K. C., Pule M A, Yotnda P et al. An inducible caspase 9 safety switch for T-cell therapy. Blood 2005; 105:4247-4254.
Study of Administration of CMV-specific Cytotoxic T Lymphocytes Expressing CAR Targeting HER2 in Patients With GBM (HERT-GBM). 2011.
Till, B. G., Jensen M C, Wang J et al. Adoptive immunotherapy for indolent non-Hodgkin lymphoma and mantle cell lymphoma using genetically modified autologous CD₂O-specific T cells. Blood 2008; 112:2261-2271.
Tlsty, T. D., Coussens L M. Tumor stroma and regulation of cancer development. Annu. Rev. Pathol. 2006; 1:119-150.
Vera, J., et al. T lymphocytes redirected against the kappa light chain of human immunoglobulin efficiently kill mature B lymphocyte-derived malignant cells. Blood 108, 3890-3897 (2006).
Verneris, M. R., et al. Low levels of Her2/neu expressed by Ewing's family tumor cell lines can redirect cytokine-induced killer cells. Clin. Cancer Res. 11, 4561-4570 (2005).
Walter, E. A., Greenberg P D, Gilbert M J et al. Reconstitution of cellular immunity against cytomegalovirus in recipients of allogeneic bone marrow by transfer of T-cell clones from the donor. N Engl J Med 1995; 333:1038-1044.
Weijtens, M. E., Hart, E. H., & Bolhuis, R. L. Functional balance between T cell chimeric receptor density and tumor associated antigen density: CTL mediated cytolysis and lymphokine production. Gene Ther. 7, 35-42 (2000).
Wels, W. et al. Construction, bacterial expression and characterization of a bifunctional single-chain antibody-phosphatase fusion protein targeted to the human erbB-2 receptor. Biotechnology (N.Y.) 10, 1128-1132 (1992).
Wilkinson, I. C. et al. High resolution NMR-based model for the structure of a scFv-IL-1beta complex: potential for NMR as a key tool in therapeutic antibody design and development. J. Biol. Chem. 284, 31928-31935 (2009).
Wodak, S. J. From the Mediterranean coast to the shores of Lake Ontario: CAPR1 's premiere on the American continent. Proteins 69, 697-698 (2007).
Wu, C., et al. Simultaneous targeting of multiple disease mediators by a dual-variable-domain immunoglobulin. Nat. Biotechnol. 25, 1290-1297 (2007).
Yamamoto, T., et al. Similarity of protein encoded by the human c-erb-B-2 gene to epidermal growth factor receptor. Nature 319, 230-234 (1986).
Yee, C., Thompson J A, Byrd D et al. Adoptive T cell therapy using antigen-specific CD8+ T cell clones for the treatment of patients with metastatic melanoma: in vivo persistence, migration, and antitumor effect of transferred T cells. Proc. Natl. Acad. Sci. U.S.A 2002; 99:16168-16173.
Zhou, X., Vink, M., Klayer, B., Berkhout, B., & Das, A. T. Optimization of the Tet-On system for regulated gene expression through viral evolution. Gene Ther. 13, 1382-1390 (2006).
Zola, H., et al. Preparation and characterization of a chimeric CD19 monoclonal antibody. Immunol. Cell Biol. 69 (Pt 6), 411-422 (1991).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A cell comprising a chimeric antigen receptor (CAR) comprising two or more non-identical antigen recognition domains.

2. The cell of claim 1, wherein the CAR is further defined as comprising an exodomain comprising an antigen recognition domain specific for a first tumor antigen and an antigen recognition domain specific for a second tumor antigen.

3. The cell of claim 1, wherein the two or more antigens are configured in the CAR in a tandem arrangement.

4. The cell of claim 2, wherein at least one of the first and second tumor antigens is specific for an antigen present on a cancer cell surface.

5. The cell of claim 4, wherein the first or second tumor antigen is specific for HER2, CD19, IL13R-alpha2, Tem8, MUC1, PSMA or EphA2.

6. The cell of claim 2, wherein at least one of the first and second tumor antigens is specific for an antigen present in a tumor microenvironment.

7. The cell of claim 6, wherein the first or second tumor antigen is specific for VEGF-A, Tem8 or FAP.

8. The cell of claim 2, wherein the first tumor antigen is specific for an antigen present on a cancer cell surface and the second tumor antigen is present in a tumor microenvironment.

9. The cell of claim 2, wherein the first tumor antigen, second tumor antigen, or both are specific for a growth factor.

10. The cell of claim 1, wherein there is a linker region between the two non-identical antigen recognition domains.

11. The cell of claim 10, wherein the linker region is between 5 and 30 amino acids.

12. The cell of claim 11, wherein the linker region is comprised of glycine, serine, or both.

13. The cell of claim 1, wherein the CAR further comprises a signaling endodomain of a costimulatory molecule selected from the group consisting of CD 28, 41BB, OX40 and zeta chain of the T cell receptor.

14. The cell of claim 1, wherein the two non-identical antigen recognition domains are HER2 and VEGF-A.

15. The cell of claim 1, wherein the two non-identical antigen recognition domains are HER2 and CD 19.

16. The cell of claim 1, wherein the two non-identical antigen recognition domains are selected from the group consisting of HER2, CD19, IL13R-alpha2, Tem8, FAP, EphA2 and VEGF-A.

17. The cell of claim 1, further defined as a T cell, a NK cell, or a NKT cell.

18. An expression vector encoding a CAR comprising two or more non-identical antigen recognition domains.

19. The vector of claim 18, further defined as a lentiviral vector, a retroviral vector, an adenoviral vector, an adeno-associated viral vector, a plasmid, or RNA.

20. A method of producing the cell of claim 1, comprising the step of transducing a T lymphocyte with an expression vector that encodes a CAR comprising two or more non-identical antigen recognition domains.

21. A method of killing a cancer cell in an individual, comprising the step of providing to the individual a therapeutically effective amount of cells of claim 1.

22. The method of claim 21, wherein the individual has breast cancer, lung cancer, brain cancer, prostate cancer, pancreatic cancer, ovarian cancer, colon cancer, liver cancer, thyroid cancer, skin cancer, testicular cancer, gall bladder cancer, esophageal cancer, spleen cancer, cervical cancer, or primary or secondary malignancies of the nervous system.

23. The method of claim 21, further comprising the step of delivering to the individual an additional cancer therapy.

24. The method of claim 23, wherein the additional cancer therapy comprises surgery, radiation, hormone therapy, chemotherapy, immunotherapy, or a combination thereof.

25. The method of claim 23, wherein when the CAR is specific at least for HER2, the individual is provided an additional HER2 therapy.

26. The method of claim 23, wherein when the CAR is specific at least for VEGF-A, the individual is provided an additional VEGF-A therapy.

27. A kit comprising cells comprising a chimeric antigen receptor (CAR) comprising two or more non-identical antigen recognition domains and/or expression vector encoding a CAR comprising two or more non-identical antigen recognition domains.
